(19) United States
(12) Patent Application Publication
(10) Pub. No.: US 2005/0164306 A1
(43) Pub. Date: Jul. 28, 2005

(54) Methods for screening for a compound useful in the treatment or prevention of lymphocytic disorders, for inhibiting lymphocite activity and preventing or treating lymphocytic disorders

(76) Inventors: Adrien Beaudoin, Rock Forest (CA); Ouhida Benrezzak, Rock Forest (CA)

Correspondence Address: GOUDREAU GAGE DUBUC, 800 PLACE VICTORIA, SUITE 3400, MONTREAL, QUEBEC, H4Z 1E9, CA

(21) Appl. No.: 10/511,133

(22) PCT Filed: Apr. 22, 2003
(86) PCT No.: PCT/CA03/00583

(30) Foreign Application Priority Data
Apr. 19, 2002 (CA) 2,382,768

(51) Int. Cl. G01N 33/53; G01N 33/567; A61K 31/7076

(52) U.S. Cl. 435/7.2; 514/45

(57) ABSTRACT

A method of screening for a compound useful in the treatment of a disease or condition characterized by an immune cells disorder, wherein said cell expresses NTPDases, said method comprising the steps of contacting a candidate compound with ecto-nucleoside triphosphate diphosphohydrolase (NTPDase), wherein the candidate compound is selected if the activity of said NTPDase is reduced in the presence of the candidate compound as compared to that in the absence thereof. A method for inhibiting an immune cell activity in a mammal, comprising targeting immune cells with an effective amount of a NTPDase inhibitor. A method to prevent or reduce the risk of rejection of transplanted tissue or organ, comprising administering to said animal an effective amount of NTPDase inhibitor.

### FIELD OF THE INVENTION

The present invention relates to methods for screening for a compound useful in the treatment or prevention of lymphocytic disorders, for inhibiting lymphocyte activity and preventing or treating lymphocytic disorders. More specifically, the present invention is concerned with NTPDase inhibitors and methods of using same.

### BACKGROUND OF THE INVENTION

Many conditions and disease involve defective or undesirable T lymphocyte proliferation or activation; these disease and conditions include graft-host reaction such as organ or tissue rejection, neoplasia, tumors, T cell leukemia, immune diseases and various diseases and conditions in oncology and oncohematology, etc.

In particular, the T cell leukemia is a T lymphocyte disorder: a progressive or aggressive disease. The T cell malignancies can be classified into two principal groups: 1-T cell acute lymphoblastic leukemia (ATLL) and 2-T cell chronic lymphoblastic leukemia.

Actual clinical therapies include chemotherapy, radiotherapy, bone marrow transplantation, or a combination of this treatment. Patients are almost always resistant to chemothereupic agent and were refractory to other therapies. ATLL is a very aggressive T cell malignancy for which no successful treatment is yet available. The acute forms have a poor outcome with overall survival ranging from 5 to 13 months. ATLL does not respond or only transiently to combination chemotherapy.

Patients with chronic forms become refractory to therapy. The poor response to therapy related to chemotherapy resistance. Results are not superior with immunotherapy.

Adenosine triphosphate (ATP) and Adenosine diphosphate (ADP) act as signaling molecules for cells of virtually all origins. Indeed these purines, which are released from the cells by exocytotic and non-exocytotic mechanisms can interact with specific receptors and thereby can influence the different physiological systems. One finds on the cell surface two classes of receptors which respond to ATP and ADP and identified as purinoceptors, and one additional class of purinoceptors which respond to adenosine. In the immune system, the role of purines is still poorly understood. It is well known however that extracellular ATP can modulate certain responses of various lymphocyte cell polulations such as DNA synthesis, blastogenesis and mediated cell killing via specific cell membrane purinoceptors. In these respects, it has been shown that ATP inhibits natural killer cell activity (11) of human and murine origins and phagocytosis in mouse macrophages (3). Extracellular ATP can stimulate in vivo DNA synthesis of thymocytes but inhibits DNA synthesis of spleen and peripheral blood lymphocytes (6). Baricordi et al (7), reported that AT had a synergistic effect on DNA synthesis stimulated by selective T-cell mitogens such as PHA or anti-CD₃ monoclonal antibody.

At least two functionally distinct purinoceptors subtypes have been described in lymphotytes: 1) that coupled to G proteins, namely the P2Y receptor linked to IP3 generation and Ca2+ mobilization from intracellular stores and 2) the P2X/P2Z receptor which gates a channel permeable to Na+ and Ca2+ (7). A P2Y receptor has been described in human B lymphocytes and has been reported to be absent from T cells (13-17). Whereas nucleotide activated ion channel has been shown to be expressed to a low level in normal B-lymphocytes and to be upregulated in resting mouse T and B-lymphocytes, leukemic peripheral blood lymphocytes. According to Baricordi et al. (7), human peripheral blood lymphocytes and purified T lymphocytes express a P2×7 purinoceptor and ionic channel gated by extracellular ATP that is involved in the control of mitogenic stimulation by different stimuli. The extracellular concentration of the agonist (ATP, ADP or adenosine) which elicits the cellular response is determined by several parameters: 1) rate of release and diffusion, ii) metabolism by ectonucleotidases and iii) binding affinity of the receptors. In this context, the role played by ectonucleotidases, and more specifically ectonucleoside triphosphate diphosphorylase (NTPDase), appears to be determinant. In this respect, immuno competent cells, such as lymphocytes and macrophages, express ectonucleotidases activities. Ectonucleotidase which catalyses the hydrolysis of ATP to ADP has been reported to B cells (18), macrophages (20), natural killer (NK) cells and CTL (21). It has been proposed that an ecto ATPase could protect murine CTL cells from the lytic effects of extracellular ATP released during granules exocytosis (1, 10, 22) and that this ecto ATPase was required for the cytolytic activity of NK cells. Recent data on ATPDase show that NTPDase 1 (CD₃₉), which is distributed on the cell surface of many cell types, plays a key role in the conversion of extracellular ATP to ADP and ADP to AMP. This enzyme, put in evidence many years ago by Lebel et al. (36) in pig pancreas, has been recently identified in primary and secondary lymphoid organs including spleen, thymus, tonsils, and Peyer's patches and isolated lymphocytes and macrophages from pig spleen (Benrezzak et al. (1999)). It has also been demonstrated that CD₃₉, a lymphoid cell activation antigen (30), corresponds to human NTPDase (31). Kansas et al. (32) cloned the latter molecule and studied its distribution and they reported that this protein is expressed on activated NK, B, and T cells of peripheral blood and is found in certain lymphoid tissues, namely tonsils and thymus. Despite these reports demonstrating the presence of NTPDase (CD₃₉) in the immune system, the physiological role played by the NTPDase in the immune response remains to be clarified. A relevant obstacle to the understanding of the NTPDase functions is a lack of specific inhibitors, i.e. an inhibitor that does not interfere with purinoceptors. A recently described NTPDase inhibitor, BGO 136, a naphtyl derivative also known as 1-hydroxynaphtalene-3,6 disulfonic acid, produced a mixed type of inhibition with Kiₛ of 380 uM with ATP and ADP as substrate, respectively (Gendron et al. 2001). Biochemical and pharmacological characterisations of a further NTPDase inhibitor, the 8 Bus ATP, are described in Gendron et al. (2000), J Med Chem. The U.S. patent application Ser. No. 09/591,177 in the name Beaudoin et al. teaches that various others compounds are also able to inhibit NTPDase activity.

There remains a need to determine the activity and functions of the NTPDase. In particular, there remains a need to determine whether NTPDase has a direct implication in immune responses.

There also remains a need to provide new means of preventing or treating diseases or conditions involving defective or undesirable T lymphocyte proliferation or activation.

### SUMMARY OF THE INVENTION

The present invention teaches that NTPDase is directly involved in the immune response. It teaches that NTPDase is an agonist of T lymphocyte proliferation and humoral response. It also teaches that NTPDase inhibitors are able to inhibit T lymphocyte proliferation and humoral response. More specifically, it teaches that NTPDase inhibitors may be used to prevent graft rejection.

More specifically, the present invention also teaches the inhibitory effects of BGO 136, erythrosin B, 8 Bus-ATP, 8 Bus-AMP on cell specific mitogenic stimulation and lymphoproliferative properties of T cells and on primary antibody response to a T-cell dependent antigen. These findings emphasize the importance of NTPDase for the T-helper cell for functions in humoral responses induced by T cell dependent antigens.

Results obtained on mouse revealed efficacy of NTPDase inhibitors and tolerance for long-term treatment.

The present invention provides uses and methods of using NTPDase inhibitors in the prevention or treatment of disease or condition characterized by a T lymphocytic disorder including graft vs host reaction following organ or tissue transplant, blood disorders such as neoplasia (leukemia and lymphoma), autoimmune disorders such as rheumatoid arthritis and psoriasis, and various other oncological and oncohematological diseases and conditions.

Composition within the scope of the present invention should contain the NTPDase inhibitor in an amount effective to achieve the desired therapeutic effect while avoiding adverse side effects. The dosage will be adapted by the clinician in accordance with conventional factors such as the extent of the disease and different parameters from the patient. Typically, 0.001 to 50 mg/kg/day of the NTPDase inhibitor in accordance with the present invention will be administered to the mammal. Pharmaceutically acceptable preparations and salts of the NTPDase inhibitor are within the scope of the present invention and are well known in the art (Remington's Pharmaceutical Science, 16th Ed., Mack Ed.).

Assays to Identify NTPDase of the Present Invention

Preferred methods for testing the ability of candidate compounds to inhibit the NTPDase activity are presented herein. It will be understood that the invention is not so limited. Indeed, often assays well known in the art can be used in order to identify non-competitive, extracellular antagonists of the present invention.

It shall be understood that the “in vivo” experimental model can also be used to carry out an “in vitro” assay.

In Vitro Assays

In a preferred embodiment, candidate inhibitors are tested for their ability to inhibit NTPDase ability to modulate lymphocytic proliferation with the incorporated triated thymidine method. Other ways of determine cellular proliferation may be use. For instance cell count and the assays described in Baker F. L. et al. (1995) Cell Prolif. 28(1): 1-15; Cheviron N. et al. (1996) Cell Prolif. 29(8): 437-46; Hu Z. W. et al. (1999) J: Pharmacol. Exp. Ther. 290(1): 28-37; and Elliott K. et al. (1999) Oncogene 18(24): 3564-73.

In Vivo Assays

The assays described above may be used as initial or primary screens to detect promising lead compounds for further development. Lead inhibitors will be further assessed in additional, different screens. Therefore, this invention also includes secondary screens which may involve various assays utilizing mammalian cell lines expressing these purinoceptors or other assays.

In yet other preferred embodiments, candidate inhibitors are tested for their ability to inhibit NTPDase's ability to elicit humoral response and specifically antibody production. Any methods known in the art including those described herein for determining antibody production may be used including the techniques involving the formation of hemolytic plaques, etc.

Tertiary screens may involve the study of the identified inhibitors in animal models for clinical symptoms. Accordingly, it is within the scope of this invention to further use an inhibitor identified as described herein in an appropriate animal model such as a rat or a mouse. For example, a inhibitor can be used in an animal model to determine the efficacy, toxicity, or side effects of treatment with such an agent. Alternatively, an inhibitor identified as described herein can be used in an animal model to determine the mechanism of action of such an agent. Furthermore, this invention pertains to uses of novel agents identified by the above-described screening assays for treatment (e.g. treatments of different types of disorders associated with a deregulated, defective, undesirable immune cells activity), as described herein.

More specifically, in accordance with the present invention, there is provided a method of screening for a compound useful in the treatment of a disease or condition characterized by an immune cells disorder, wherein said cell expresses NTPDases, said method comprising the steps of contacting a candidate compound with ecto-nucleoside triphosphate diphosphohydrolase (NTPDase), wherein the candidate compound is selected if the activity of said NTPDase is reduced in the presence of the candidate compound as compared to that in the absence thereof. In a specific embodiment, the contacting of the candidate compound with the NTPDase is performed in an immune cell selected from the group consisting in normal T lymphocyte, normal B lymphocyte, normal NK cell, normal macrophage, normal monocyte, Jurkat cell, Raji cell, Ramos cell, MonoMac™ cell, K562 cell and U937 cell.

According to a further aspect of the present invention, there is provided a method for inhibiting an immune cell activity in a mammal, comprising targeting immune cells with an effective amount of a NTPDase inhibitor. In specific embodiments, the method is further characterized by one or more of the following characteristics: the immune cells are normal lymphocytes (such as T and B lymphocytes) or tumoral immune cells (such as T or B neoplastic lymphocytes, and more specifically Jurkat cells), the activity is the T lymphocyte proliferation or the production of antibodies. In more specific embodiments, the activity is induced either by organ or tissue transplant, by an allergen, or by autoimmune diseases. In further specific embodiments, the NTPDase inhibitor is selected from the group consisting of BGO 136, erythrosin B, nucleotide or nucleotide derivative including AMP, 8 Bus-AMP and 8 Bus-ATP, and analogues thereof.

In a further aspect of the present invention, there is provided a method to prevent or reduce the risk of rejection of transplanted tissue or organ, comprising administering to the mammal an effective amount of NTPDase inhibitor. In a specific embodiment, the NTPDase inhibitor is selected from the group consisting of BGO 136, erythrosin B, nucleotide or nucleotide derivative including AMP, 8 Bus-AMP and 8 Bus-ATP, and analogues thereof.

In a further aspect of the present invention, there is provided a composition for use as an immunosuppressive agent in graft transplant comprising an effective amount of BGO 136 or BGO 136 analogue in a pharmaceutically acceptable carrier.

In a further aspect of the present invention, there is provided a use of a BGO 136 or BGO 136 analogue in the making of a medicament for use as an immunosuppressive agent in graft transplant.

NTPDase is a family of enzymes which catalyse the sequential hydrolysis of the gamma and betaphosphate bonds of nucleoside tri and diphosphates and characterized by homologous sequences (apyrase conserved regions). As used herein, the term “NTPDase” refers to any member of this family.

As used herein, the term “disease or condition characterized by an immune cell disorder” is meant to include disease and conditions characterized by defective or undesirable immune cell activity. It includes normal T lymphocytes proliferation and normal B lymphocytes humoral response, any dysfunction in normal immune cells including monocytes, NK cells, and lymphocytes, neoplastic immune cells proliferation, etc. Without limiting the generality of the foregoing, such diseases and conditions include graft vs. host reaction following organ or tissue transplant (including graft rejection), blood disorders such as neoplasia (leukemia and lymphoma), autoimmune disorders including rheumatoid arthritis, psoriasis, Crohn disease and certain diabetes, oncological and oncohematological diseases and conditions.

As used, the terms “neoplastic”, and “tumoral” are used interchangeably.

As used herein, the term “NTPDase activity” is meant to include the enzyme's ability to hydrolyse ATP and ADP and any downstream effect of this enzymatic reaction including lymphocyte proliferation and humoral response.

As used herein, the term “lymphocytes” is meant to include normal lymphocytes and neoplastic/tumoral lymphocytes. Without limiting the generality of the foregoing, this term is meant to include normal T and B lymphocytes such as those found in normal mammals, and neoplastic/tumoral T and B lymphocytes such as Jurkat, Rami and Ramos cells.

As used herein, the term “effective amount” is meant to refer to an amount of NTPDase inhibitor administered in a single dose or in multiple doses sufficient to reduce or abrogate the undesirable lymphocyte activity in the cell or the animal to which it is administered while avoiding adverse side effects. Without limiting the generality of the foregoing, the results presented herein demonstrate that an amount of 4,3 mg/g of base weight of transplanted spleen is sufficient to reduce the spleen increase by 56%. It refers to the amount necessary to produce a benefit to the cell or animal to which it is administered as determined by a person of ordinary skill in the art.

As used herein, the term “BGO 136 analogue” is meant to refer to any naphtyl derivative having the ability to inhibit an NTPDase activity. Furthermore, the F. B. Gendron doctorate thesis submitted to the University of Sherbrooke on inhibitors defines the conformational characteristics that enable an analogue to possess inhibitory abilities.

As used herein, the term “AMP analogue” is meant to refer to any adenosine 5′-monophosphate derivative having the ability to inhibit an NTPDase activity including 8-BUS AMP. Since AMP is the last member of the reaction, it is submitted that it is reasonably predictable that any AMP analogue will inhibit NTPdase.

As used herein, the term “ATP analogue” is meant to refer to any adenosine 5′-triphosphate derivative having the ability to inhibit an NTPDase activity including 8-BUS ATP. Gendron et al (2000a) and Nahum et al (2002) describe such ATP analogues.

As used herein, the term “erythrosin B” is meant to refer to any member of the erythrosin family or erythrosin derivative having the ability to inhibit an NTPDase activity.

As used herein, the term “nucleotide derivative” is meant to refer to any nucleotide having been subjected to any modification preserving its ability to inhibit an NTPDase activity. Assays as described in Picher et al (1996) showed that modifications on the ATP phosphate chain prevent ATP hydrolysis consequently granting resulting molecules with inhibitory activities.

As used herein, the term “humoral response” is meant to include antibody production.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 shows the presence (the 78 KDa and 56 KDa lines) of NTPDase in human lymphoid cells. In this figure, PBL: normal peripheral blood cells; Ramos: B leukemia cells; Jurkat: T leukemia cells; K562: erytroleukemia cells; Raji: B leukemia cells; NK: natural killer cells; Monomac™: monocytes leukemia cells;

FIG. 2 shows the effect of BGO 136 on NTPDases activity of human peripheral blood lymphocytes (PBL). Panels A and B show BGO 136's effect on the ATPDase and ADPDase functions, respectively. Eight (8) assays were performed each in triplicate. NTPDase 1 and 2 (in a lesser extent) were used;

FIG. 3 shows the inhibitory effect of BGO 136 on human peripheral blood lymphocytes (PBL) proliferation. Panel A presents results with a control (unstimulated cells); Panel B presents results with cells stimulated by ConA™ and Panel C presents results with cells stimulated with LPS™. Three (3) experiments were performed each in quadruplicate. [inhibitor]: indicates inhibitor concentration;

FIG. 4 shows the correlation between NTPDase activity of cells subjected to BGO 136 as presented in FIG. 2 and proliferation assay of peripheral blood lymphocytes (PBL) subjected to BGO 136 as presented in FIG. 3. Each set of point corresponds to a single concentration of the inhibitor (concentration used are 0, 1, 5 and 10 mM of BGO136);

FIG. 5 shows the effect of BGO136 on humoral response (antibodies production) 21 days after a first injection (Panel A), 32 days after first injection (Panel B); and 40 days after a first injection (Panel C). In this figure, DM: indicates that multiple doses of BGO136 were given; +Ag: mice received BSA as Ag T cell dependent; −Ag: mice received only vehicle; BGO136 was given in a single dose at the mentioned concentration (400 or 800 mM). Each group contained ten (10) mice;

FIG. 6 shows the effect of erythrosin B on NTPDase activity of human peripheral blood lymphocytes (PBL). Six (6) experiments were performed each in triplicate;

FIG. 7 shows the effect of erythrosin B on human peripheral blood lymphocytes (PBL) proliferation. Three (3) experiments were performed each in sextuplicate;

FIG. 8 shows the correlation between NTPDase activity of cells subjected to erythrosin B as presented in FIG. 6 and proliferation assay of peripheral blood lymphocytes (PBL) subjected to erythrosin B as presented in FIG. 7. Each set of point corresponds to a single concentration of the inhibitor. Concentrations used are 0, 10, 30, 50 and 100 uM of erythrosin B;

FIG. 9 shows the effect of erythrosin B on humoral response (antibody production) 21 days after first injection (Panel A), 32 days after first injection (Panel B) and 40 days after first injection (Panel C). In this figure, DM Multiple doses of Erythrosin B were given; Groupe Ag: mice received only BSA as Ag T cell dependant without inhibitor; Erythrosin B was given in a single dose as mentioned 0.25 g/kg or 0.5 g/kg. Each group contain 10 mice;

FIG. 10 shows the inhibitory effect of 8-BuS ATP on NTPDase activity of human peripheral blood lymphocyte. One (1) experiment was performed in duplicate;

FIG. 11 shows the inhibitory effect of 8-BuS-ATP on the human peripheral blood lymphocytes (PBL) proliferation. Three (3) experiments were performed each in sextuplicate;

FIG. 12 shows the correlation between NTPDase activity of cells subjected to 8-BuS-ATP as presented in FIG. 10 and proliferation assay of peripheral blood lymphocytes (PBL) subjected to 8-BuS-ATP as presented in FIG. 11. Each set of point corresponds to a single concentration of the inhibitor. Concentrations used are 0, 10, 50 and 100 uM of 8-BuS-ATP;

FIG. 13 shows the inhibitory effect of 8-BuS AMP on NTPDase activity of human peripheral blood lymphocyte. Two (2) experiments were performed each in triplicate;

FIG. 14 shows the inhibitory effect of 8-BuS-AMP on the human peripheral blood lymphocytes (PBL) proliferation. Three (3) experiments were performed each in sextuplicate;

FIG. 15 shows the correlation between NTPDase activity of cells subjected to 8-BuS-AMP as presented in FIG. 13 and proliferation assay of peripheral blood lymphocytes (PBL) subjected to 8-BuS-AMP as presented in FIG. 14. Each set of point corresponds to a single concentration of the inhibitor. Concentrations used are 0, 10, 30 and 100 uM of 8-BuS-AMP;

FIG. 16 shows the inhibitory effect of beta-gamma methylene ATP on NTPDase activity of human peripheral blood lymphocytes (PBL). Two (2) experiments were performed each in triplicate;

FIG. 17 shows the inhibitory effect of beta-gamma methylene ATP on the human peripheral blood lymphocytes (PBL) proliferation. Three (3) experiments were performed each in sextuplicate;

FIG. 18 shows the correlation between NTPDase activity of cells subjected to beta-gamma methylene ATP as presented in FIG. 16 and proliferation assay of peripheral blood lymphocytes (PBL) subjected to beta-gamma methylene ATP as presented in FIG. 17. Each set of point corresponds to a single concentration of the inhibitor. Concentrations used are 0, 50, 100 and 250 uM of beta-gamma Me-ATP; and

FIG. 19 shows the inhibitory effect of NTPDase inhibition on neoplastic T cell growth Results obtained with erythrosin B, BGO 136, AMP, beta-gamma methylene ATP, 8-BuS-ATP are shown in Panels A to E, respectively. For each inhibitor, two (2) experiments were performed each in quadruplicate.

### DESCRIPTION OF SPECIFIC EMBODIMENTS

### Isolation of Human Lymphocytes

Human lymphocytes were obtained from peripheral venous blood of normal and healthy medication-free volunteers. Fresh blood collected in EDTA glass tubes was layered into Histopaque™-1077 (a solution of Ficoll™ and sodium diatrizoate adjusted to a density of 1.077, Sigma, USA) and centrifuged at 400 g for exactly 30 min, at room temperature. During centrifugation, erythrocytes and granulocytes were aggregated by Ficoll™ and rapidly sedimented, whereas lymphocytes remained at the plasma interface. Plasma (the upper layer) was carefully removed to prevent disturbance of the buffy coat. The latter fraction rich in lymphocytes was recovered with a pasteur pipette and washed twice with RPMI™ 1640 medium by centrifugation, for 10 min, at 250×g. The final pellet was suspended in fresh RPMI™ 1640 medium supplemented with 2 mM L-glutamine, 10% foetal bovine serum inactivated at 56° C. for 30 min, and antibiotics: penicillin 100 units/nl, streptomucin 100 ug/ml and amphotericin 2.5 ug/ml. Cells were counted with the hemacytometer. Viability, tested with Trypan™ blue exclusion assay, was superior to 90%. The cell preparation named PBL (for peripheral blood lymphocyte) was immediately used for NTPDase assays and for mitogenic responses to stimulators.

### Maintenance of Lymphoblastoid Human Cell Lines

Human cell lines were given by Dr. Jana Stankova (Department of Immunology, Université de Sherbrooke). Jurkat cells were derived from human T cell leukemia. Raji and Ramos cells are B-cell lymphomas derived from peripheral blood of patients with Burkitt lymphoma. MonoMac™-1 is a cell line derived from the peripheral blood of a 64 year-old male with active monocytic leukemia. K562, which is used as highly sensitive target for NK cell activity is an erythroleukemia cell line. U937 is a promonocytic myeloid cell and finally the Y2 T2 C2 (NY) cell is a human natural killer cell line. These lymphoblastoid cell lines which grow in suspension as single cells without attachment to glass, were maintained by passage in complete RPMI 1640 medium with 2 mM L-glutamine containing 10-20% heat inactivated foetal bovine serum [V/V] and antibiotics (100 units/ml of penicillin), streptomycin 100 ug/ml and 2.5 ug/ml of amphotericin) at 37° C. in a humidified 5% CO₂/95% air atmosphere. Mono-Mac™ 1 cells were maintained in the same complete RPMI™ 1640 medium supplemented with 1× non-essential amino acids and 1 mM Na-pyruvate at 37° C. with 5% CO₂.

### Mitogen Stimulation and Proliferation Assay

A thymidine uptake assay was used to evaluate the effect of NDPase inhibitors on the proliferative activity of normal lymphocytes.

Cell suspensions of fresh human peripheral blood lymphocytes (PBL) were resuspended in RPMI™ 1640 medium supplemented with 10% foetal bovine serum at a concentration of 2×106/ml. Aliquots of 100 ml were plated on a Falcon™ flask (96-well) and stimulated with 10 ug/ml of (Con-A) or 40 ug/ml of (LPS™) at a final volume of 0.2 ml/well. ConA™ and LPS™ were used to stimulate preferentially T cells and B cells, respectively. Cell cultures±inhibitors were incubated for 48 h at 37° C. in a 5% CO₂ atmosphere [³H] thymidine (1 ug/well, specific activity 5.0 ci/mmol) was added to each microplate in a volume of 50 ul. After an additional a 4 h incubation at 37° C., cells were collected with a cell harvester and ³H thymidine incorporation was measured in triplicate samples by liquid scintillation counting.

### NTPDase Assays

Prior to the assays, human normal lymphocytes as well as leukemic cell lines, were washed three times with phosphate-free saline in 95 mM NaCl, 1 mM CaCl₂, 2 mM MgCl₂ and 45 mM Tris —HCl buffer (pH 7.4). Enzyme assays were carried out at 37° C. in 1 ml of the following assay medium: 95 mM NaCl, 5 mM KCl, 1 mM CaCl2, 2 mM MgCl₂, 5 mM glucose, 0.05% BSA, 5 mM tetramisole and 45 mM Tris-HCl (pH 7.5). The reaction was started by adding the substrate (200 uM of ATP or ADP) and stopped with 250 ul of the Malachite green reagent. Controls were run with the enzyme added after the Malachite green reagent. Where indicated, 10 mM sodium azide (NaN₃) or 1 mM, 5 mM and 10 mM of BGO 136 were added directly to the assay mixture. Inorganic phosphate was estimated according to Baykov et al.⁽¹⁾ and enzyme activity was either expressed as nmoles of Pi released/min/mg of protein which corresponds to mUnits⁽²⁾ or as nmoles Pi/min/10⁶ cells. Where indicated, lymphoid cells were lysed by three freeze thaw cycles in saline buffer and protein was measured with the method of Bradford using bovine serum albumin as a standard (3).

### Electrophoresis and Western Blotting

Lymphocytes from human peripheral blood (PBL) and lymphoblastoid cell lines obtained as described above were lysed and protein was measured. Samples of 20 ug protein of whole cell lysate were applied to each well of polyacrylamide gel. Electrophoresis was carried out under denaturing conditions (SDS-PAGE) in a 10% polyacrylamide gel. Protein was subsequently transferred to immobilon-P sheets and immunoblotted. A rabbit antiserum (Kally) which recognizes isoform 11 of ATPDase was used as primary antibody at a dilution of 1:10 000. The secondary antibody, a mouse monoclonal anti rabbit IgG conjugated to alkaline phosphatase (1:10 000) was detected by chemiluminescence using the Immun-Star™ substrate, according to the recommendations of the supplier (Bio-Rad/laboratories).

### In Vivo Antibody Production Assay

Animal Treatments and Experimental Design

Immunity mice were primed with antigen by an intraperitoneal injection of BSA as a T-dependent antigen. Briefly, BSA was resuspended in PBS (pH 7.2) and emulsified in the same volume of complete Freund's adjuvant (CFA). 30 ug BSA in 100 ml PBS was given to each mouse. Four groups of ten mice were immunized.
Group 1: mice were injected i.p. with BSA alone with emulsified in (CFA) Group: Ag.
Group 2: mice were injected i.p. with BSA plus BGO 136 (400 mM) in 100 ml PBS, emulsified in (CFA) or erythrosin B (0.25 g/Kg)
Group 3: mice were injected i.p. with BSA plus BGO 136 (800 mM) in 100 ml PBS, emulsified in (CFA) or erythrosin B (0.5 g/Kg)
Group 4: mice were treated with BSA in presence of the inhibitor emulsified in (CFA). These mice received various i.p. doses of BGO 136 400 mM in 0.1 ml of normal PBS or erythrosin B 0.25 g/Kg, 2 doses/week are given as a maintenance dose. (group named DM=maintenance dose)

These initial immunizations (i.p.) were followed by two subsequent immunizations with emulsions of BSA alone or in presence of BGO136 400 mM or 800 mM, in incomplete Freund's adjuvant. Five days after each immunization, blood was collected from each animal, sera was analysed by an ELISA assay.

### Detection of Antibodies

Flat-bottomed microtiter plate were coated with BSA (5 ug/ml) in NaHCO₃/Na₂CO₃ 50 mM (pH 9.6) at 37° C. for 2 h. Plates were washed three times with (PBS buffer, pH 7.4, 0.5 ml/L Tween-20) and then blocked with 400 ul of milk per well overnight, at 4° C. Diluted sera specimens were added (100 ul/well) and incubated for 2 h at 37° C. After thorough washing, wells were incubated with 100 ul of goat-anti-mouse IgG conjugated with peroxydase for 2 h at 37° C. followed by several washes. TMB substrate solution (100 ul/well), (42 mM TMB in DMSO, 0.1 M citric acid, Na₂HPO₄ 0.2 M and H₂O₂ 30%) was added. Color development was allowed to proceed for approximately 15 to 30 min, at room temperature. Reaction was terminated by adding 30 ul of 4N H2SO₄. Optical density was read at 450 nm.

### Cell Growth and Proliferation of Jurkat Cell Lines

T cell Jurkat is an acute T cell leukemia obtained from the peripheral blood of human. It was purchassed from ATCC and was grown and maintained in RPMI™. 1640 medium containing 10% FBS, 2 mM L-glutamine adjusted to contain 1.5 g/L sodium bicarbonate, 4.5 g/L glucose, 10 mM HEPES and 1.0 mM sodium pyruvate. To evaluate the effect of the different inhibitors of NTPDases on the growth of Jurkat cells, BGO 136, erythrosin, 8 Bus ATP, AMP and β-γ-Me ATP were added in the RPMI™ 1640 medium in presence of cells at day=0. Cells counts and viability of cells were determined on day=5.

### Statistical Analysis

Mice were used as test animals. Studies were also performed on the cultures of peripheral blood lymphocytes of donors and on experimental tumor cell lines. Test results were statistically processed. A one way variance analysis was followed; the Statpack® software analysis program was utilized.
*p<0.05
** p<0.01
*** p<0.001

### EXAMPLE 1

### Presence of NTPDase in Various Normal and Neoplastic Lymphoid Cells and Inhibitory Activity of NaN3

The presence and activity of NTPDase in PBL cells and in various tumor cells were compared using ATP and ADP as substrates.

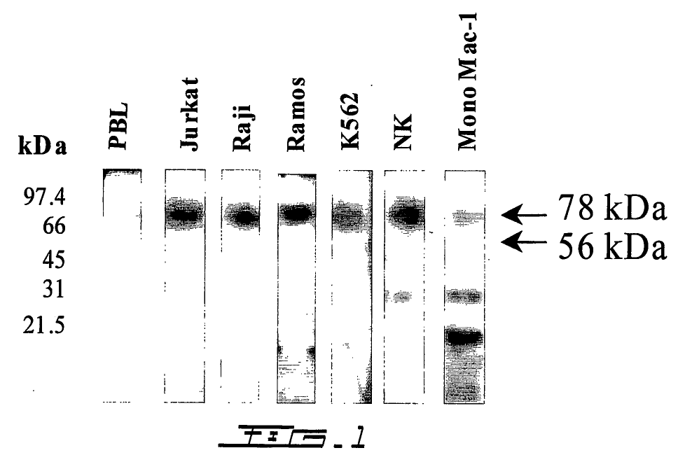

As may be seen in FIG. 1, the NTPase signal is more pronounced in neoplastic cells as compared to that in normal lymphocytes (peripheral blood lymphocytes (PBL)).

The levels of the ecto-ATPase and ecto-ADPase functions of NTPDase are presented in Table 1 below. In agreement with the Western-blots shown in FIG. 1, the levels of NTPDase activity in normal PBL cells is relatively low as compared to that in tested tumor cell lines. It is noteworthy that in most cases, 10 mM Na azide (NaN3) (a NTPDase inhibitor) causes more than 50% inhibition of enzyme activity.

TABLE 1
NTPDase activity of various intact human normal and tumoral lymphoid cells

| | | (nmoles Pi/min/10⁶ cells) | | | |
|---|---|---|---|---|---|
| | | ATP | ATP + NaN3 | ADP | ADP + NaN3 |
| n = 3 | Ramos | 20 ± 0.3*** | 10 ± 0.0 | 14.5 ± 2.4*** | 8 ± 0 |
| | Raji | 39 ± 6.0*** | 14 ± 0.0 | 21 ± 0.8*** | 14 ± 0 |
| | JurKat | 25 ± 1.5*** | 7 ± 1.0 | 9.5 ± 0.8*** | 3.5 ± 0.8 |
| | Mono Mac-1 | 23 ± 1.0*** | 7 ± 1.2 | 7.7 ± 0.2*** | 3.8 ± 1.8 |
| | U937 | 29 ± 1.0*** | 8 ± 0.0 | 11 ± 0.5*** | 3.3 ± 0.8 |
| n = 4 | PBL | 1.0 ± 0.1 | 0.5 ± 0.1 | 0.5 ± 0.1 | 0.2 ± 0.1 |

n = number of experiments (each in triplicate)
***indicates that the difference of NTPDase activity between tumor cell lines group and the control PBL group is significant.

### EXAMPLE 2

### Effect of BGO 136 on NTPDase Activity of PBL

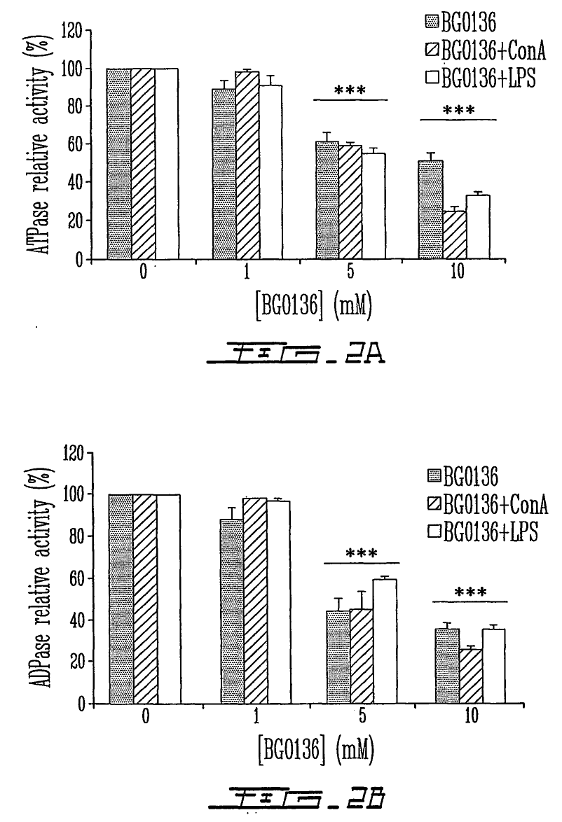

As shown in FIG. 2, with PBL, BGO 136 caused a dose-dependent inhibition of ATPase and ADPase activities. A significant inhibition (40%) was obtained with 5 mM BGO 136 whereas with 10 mM, it increased up to 50% or more. The results are expressed as percent of controls considering untreated control samples as 100.

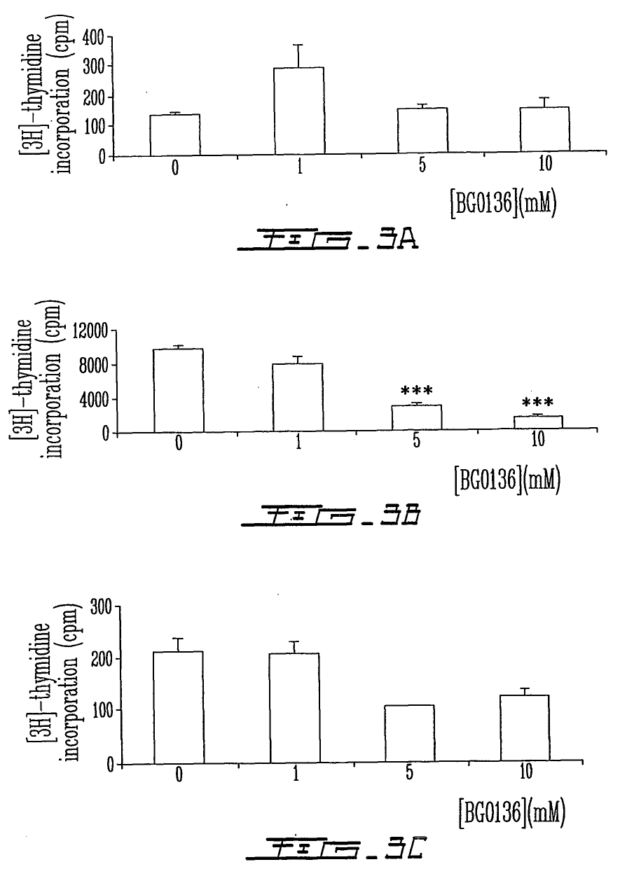

The influence of BGO 136 on human lymphocyte proliferation stimulated by ConA™ and LPS™ is shown in FIG. 3. ConA™ is a mitogen specific to lymphocytes T while LPS™ is a mitogen specific to lymphocytes B. BGO 136 did not affect the proliferation of non-activated lymphocytes as measured by 3H-Thymidine incorporation in DNA. In contrast, proliferation induced by ConA™ was inhibited in a dose-dependent manner, reaching about 10% of the positive control values with 10 mM BGO 136. The BGO 136 inhibition of LPS™-induced proliferation was much less pronounced. One has to take into account that cell-induced proliferation as expected is relatively low.

In PBL proliferation assays presented in the Examples below, ConA™ only was used to specifically stimulate T lymphocyte proliferation.

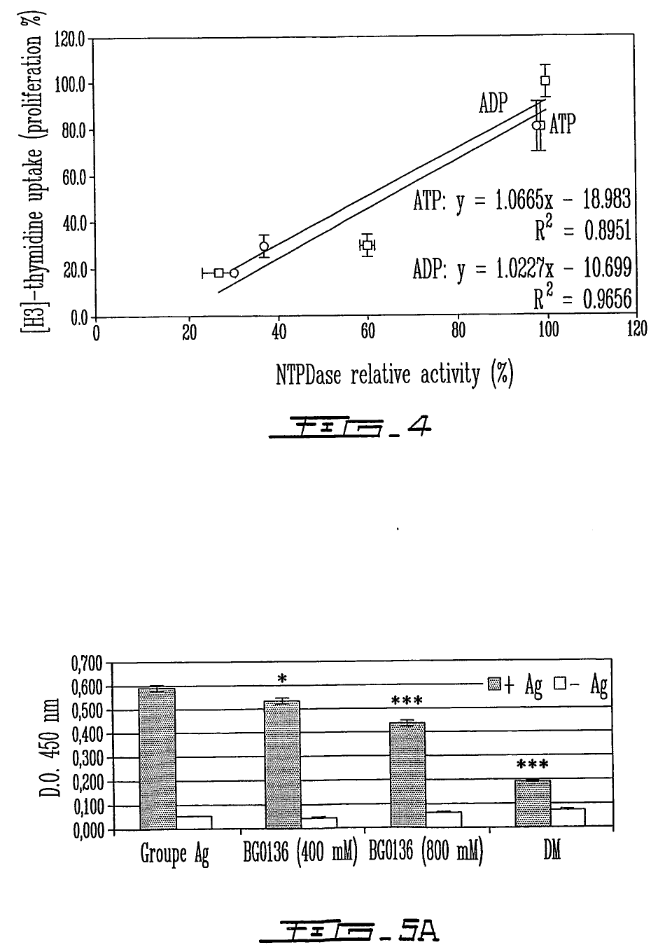

A positive correlation (0.89 for ATPase and 0.96 for ADPase) between cell proliferation presented in FIG. 3 and NTPDase activity presented in FIG. 2 was established in the presence of the three concentrations of BGO 136 is illustrated in FIG. 4.

### EXAMPLE 3

### Effect of BGO 136 on Humoral Response

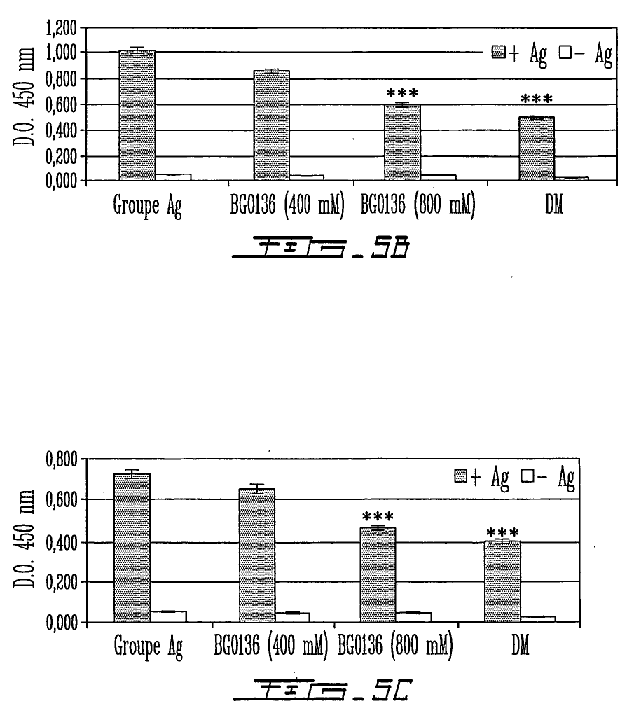

As shown in FIG. 5, the levels of albumin antibodies were reduced as much as 50% by a single dose BGO 136 (800 mM). Repeated injections of BGO 136 appeared even more efficient (group DM in FIG. 5).

The experiments were repeated in several series of mice and produced essentially the same pattern of inhibition. The results from one representative experiment are presented in FIG. 5.

No toxic or allergic reactions were observed during the BGO 136 administration (two months).

### EXAMPLE 4

### Effect of Erythrosin B on PBL NTPDase Activity and PBL Proliferation

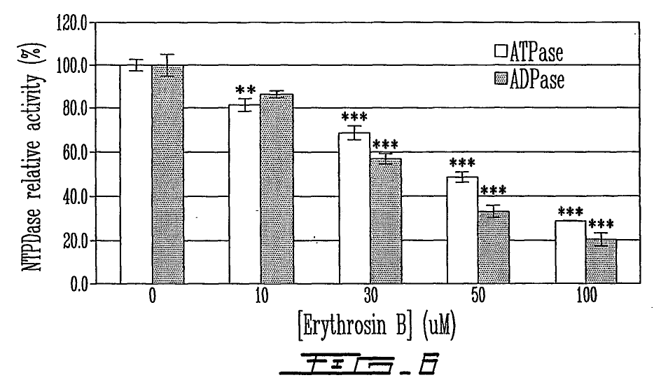

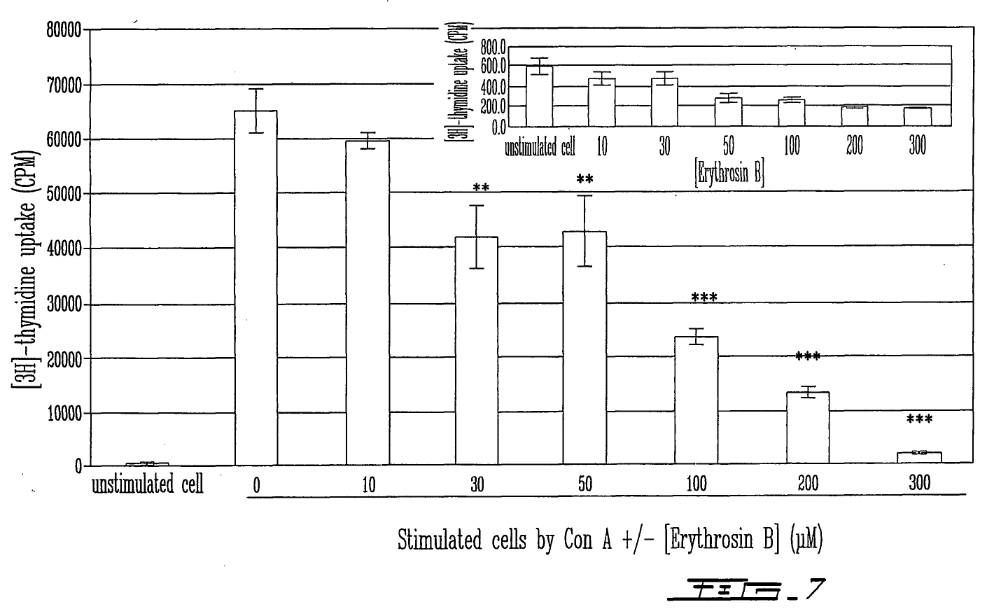

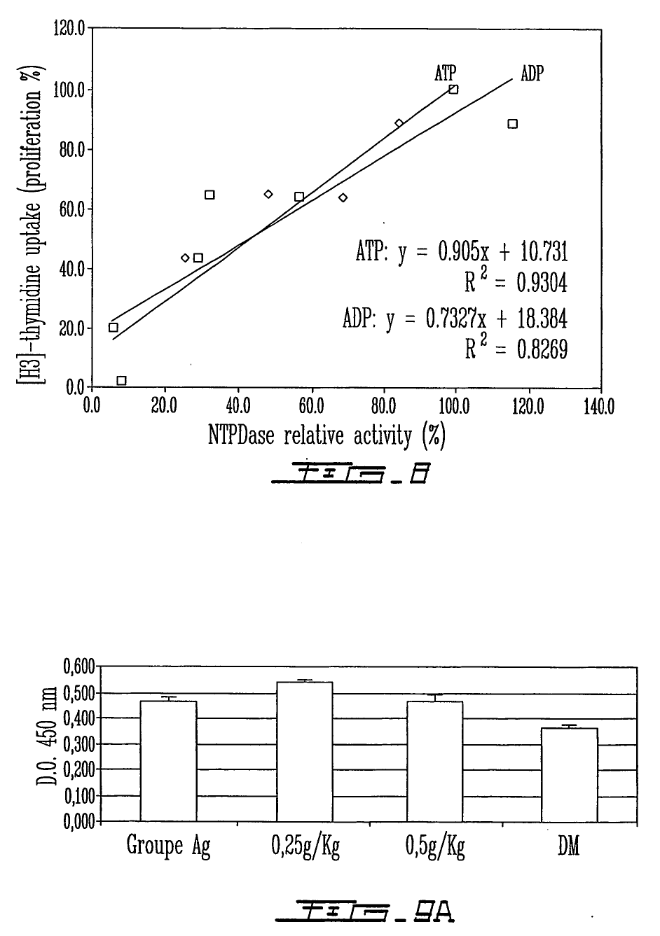

The effect of erythrosin B, a NTPDase inhibitor not structurally related to BGO 136, on lymphocyte T activity was also observed to determine its ability to influence immune response of T-cells. Results presented in FIGS. 6-7-8 show a dose-dependent inhibition of lymphocyte proliferation (FIG. 7) and NTPDase activity (FIG. 6). A high correlation was established between these two parameters (FIG. 8).

### EXAMPLE 5

### Effect of Erythrosin B on Humoral Response

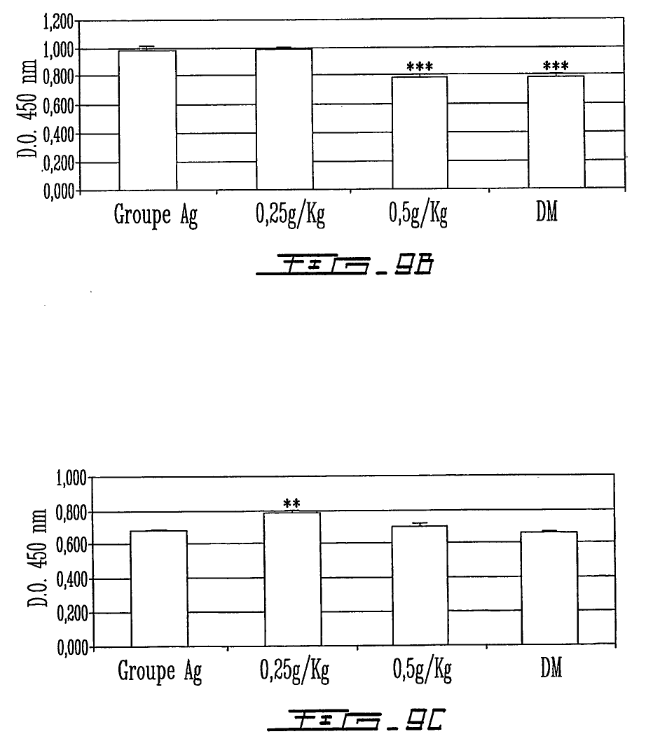

As shown in FIG. 9, a significant decrease in the levels of antibodies to albumin following administration of erythrosin B can be observed especially with multiple injections to sustain the concentration of the inhibitor in the animal (group DM in FIG. 9).

### EXAMPLE 6

### Effect of 8 BUS-ATP on PBL NTPDase Activity and PBL Proliferation

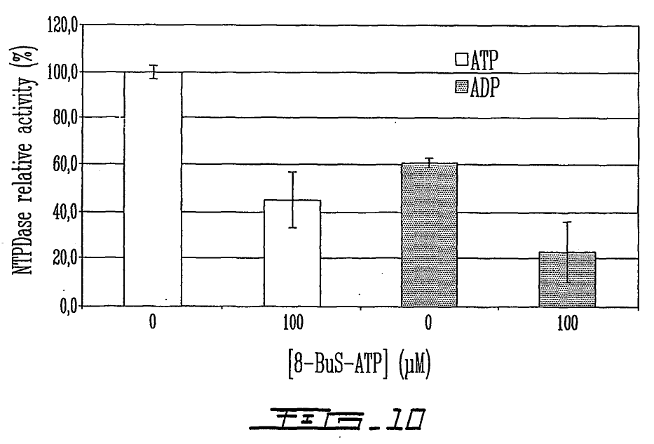

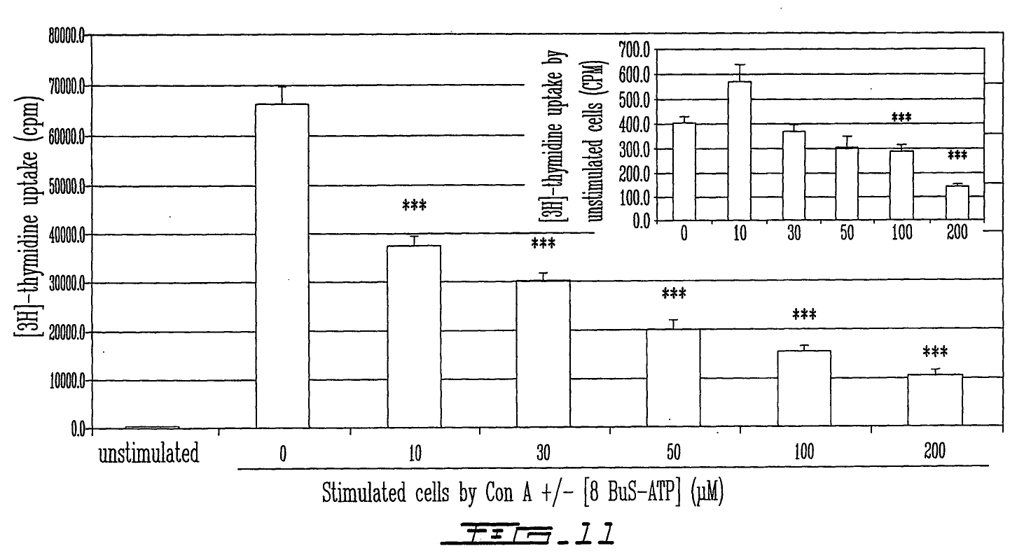

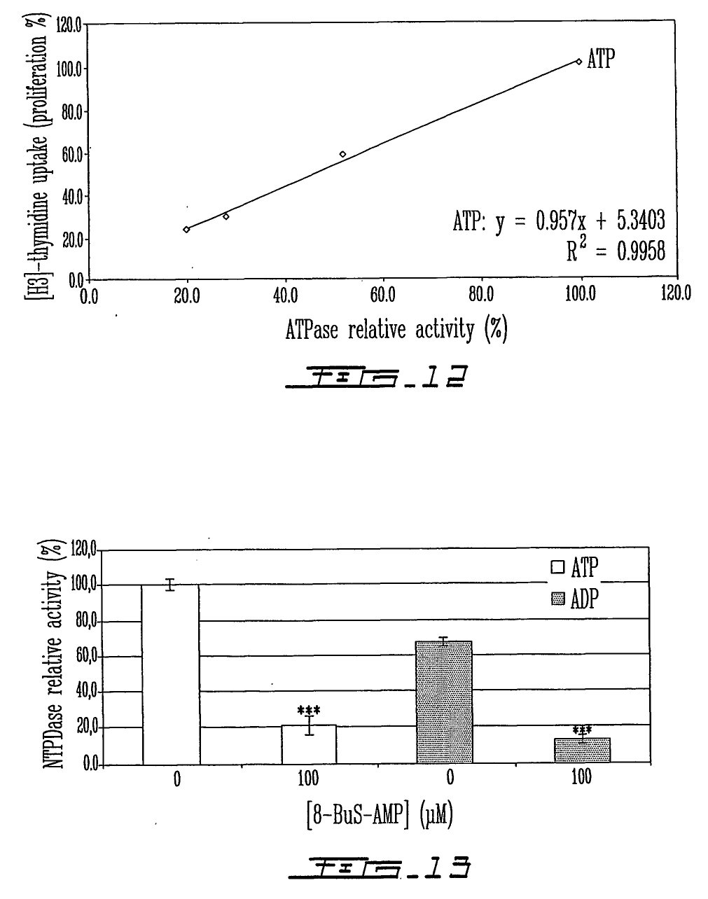

A pronounced dose-dependent inhibition of the NTPDase activity by 8 BUS-AMP was observed (FIG. 10). 8 BUS-ATP reduced cell proliferation induced by ConA™ (FIG. 11). A very good correlation could be established between these two parameters (FIG. 12).

### EXAMPLE 7

### Effect of 8 BUS-AMP on PBL NTPDase Activity and PBL Proliferation

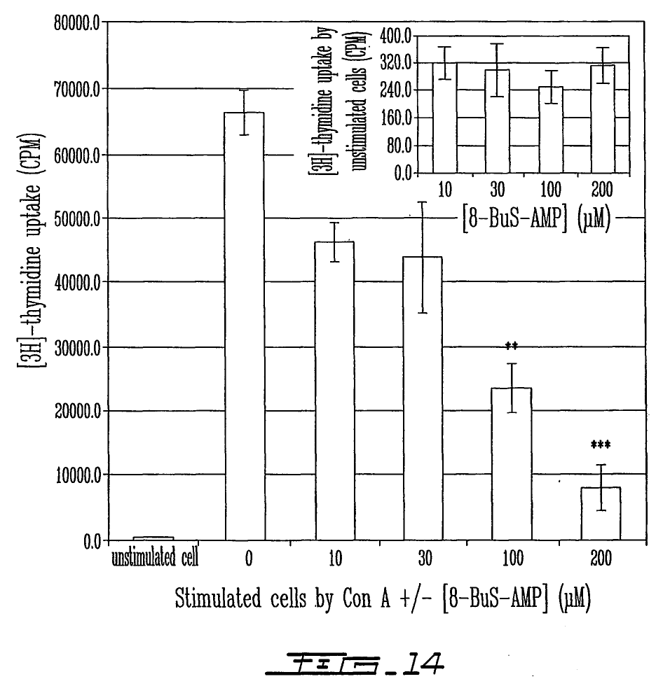

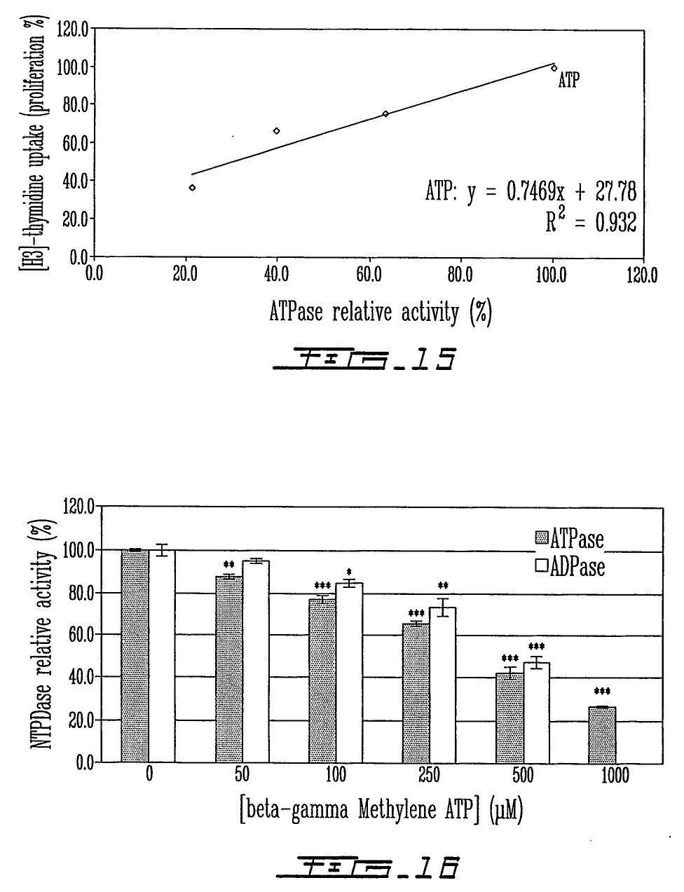

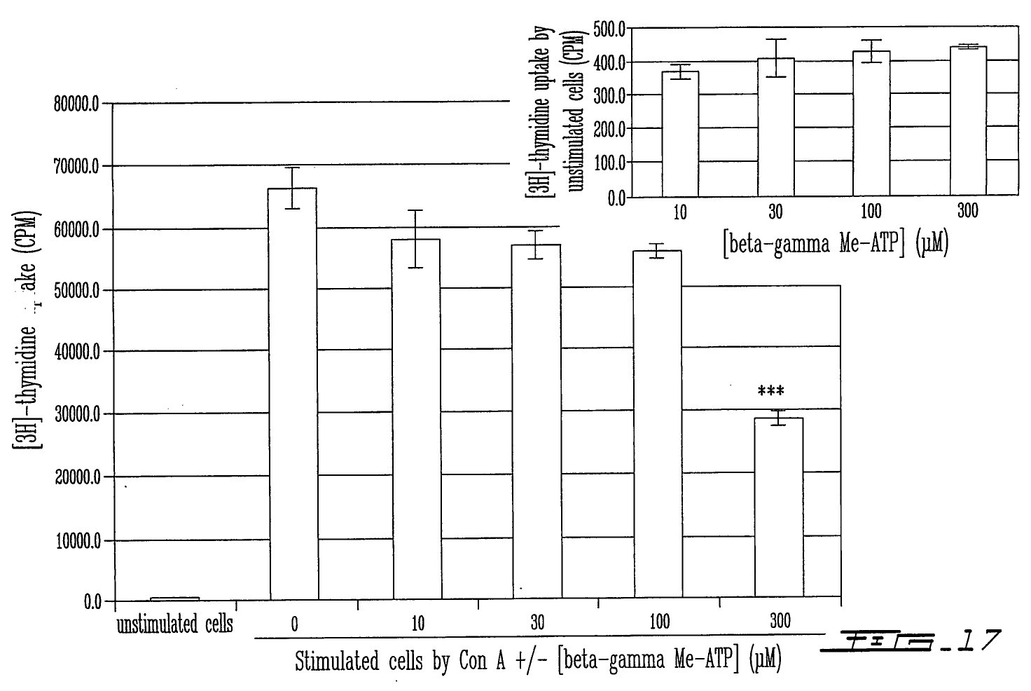

A pronounced dose-dependent inhibition of the NTPDase activity by 8 BUS-AMP was observed (FIG. 13). 8 BUS-ATP reduced cell proliferation induced by ConA™ (FIG. 14). A high correlation was established between these two parameters (FIG. 15).

### EXAMPLE 8

### Effect of β-γ-Me ATP on PBL NTPDase Activity and PBL Proliferation

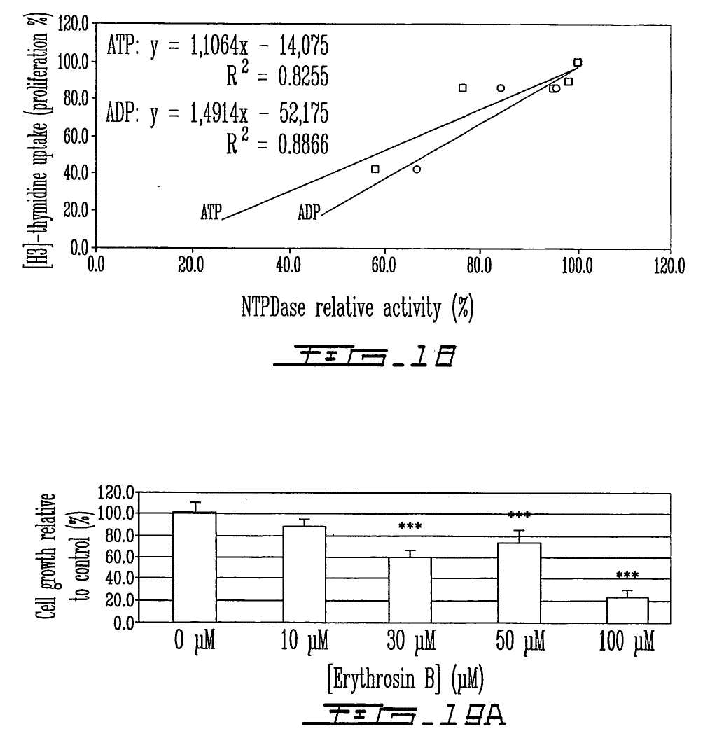

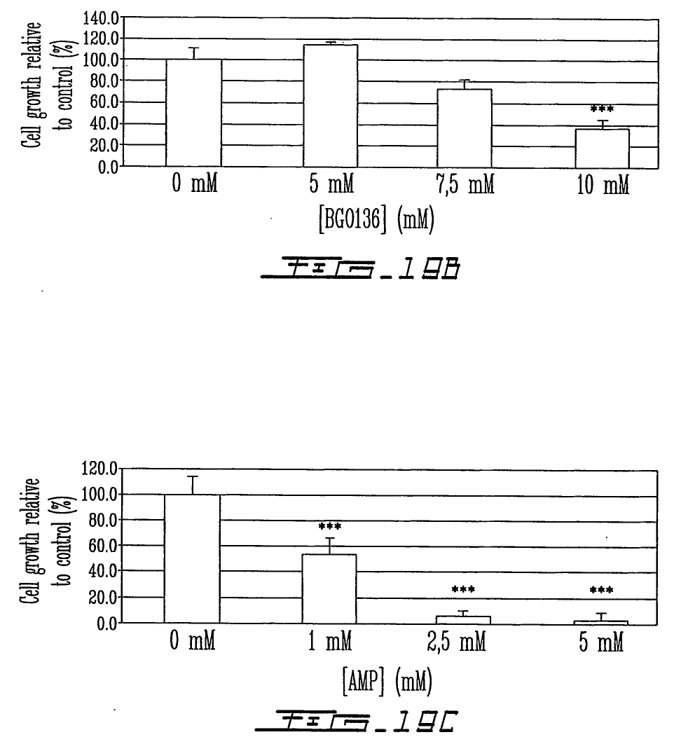

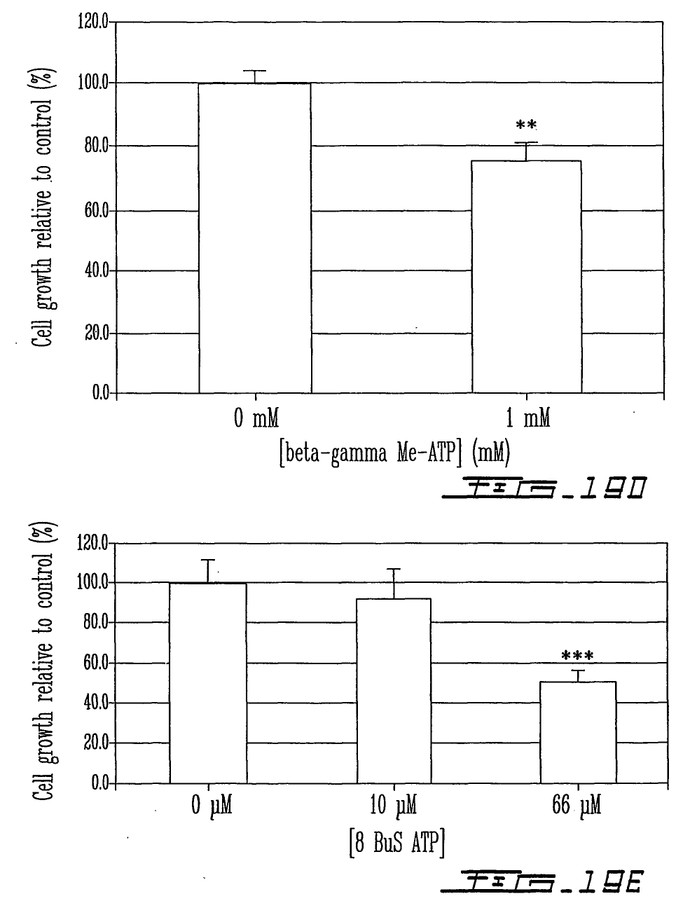

As shown in FIG. 16, NTPDase was inhibited in a dose-dependent manner. β-γ-Me ATP reduced cell proliferation induced by ConA™ (FIG. 17). Again, a high correlation was established between these two parameters (FIG. 18).

### EXAMPLE 9

### Effect of BGO 136, Erythrosin, 8 BUS ATP, AMP and β-γ-Me ATP on Jurkat Cells' Proliferation

FIG. 19 shows the influence of the different inhibitors on neoplastic T lymphocytes, namely Jurkat cells which are derived from human leukemia cells. BGO 136, erythrosin, 8 BUS ATP and AMP exerted a marked reduction of cell proliferation. A less pronounced but significant effect was observed with β-γ-Me ATP.

### EXAMPLE 10

### Comparison of Effect of NTPDase Inhibitors with Compound of the Prior Art on Graft vs. Host Reaction

Table 2 below shows the efficiency of NTPDase inhibitors in preventing mice spleen growth following cell transplant according to the Bundick model. Mice were injected their parents' spleen cells. 21 days later, their spleen was weighed. Activation of Th2 (T helper cells) cells. The treatment groups are cyclosporin, BGO, and AMP. Bold figures in Table 2 correspond to results originally obtained by Bundick. The “No treatment group” is constituted of normal mice. The “positive” group is constituted of mice having undergone spleen tissue transplantation and having been injected with the carrier only (PBS™) without inhibitor. The other groups are constituted of mice having undergone spleen tissue transplantation and having been injected with the BGO 136, AMP or the cyclosporine, respectively. The spleen of all mice was then removed and weighed. The weigh of treated mice was then compared with that of the “No treatment” mice. Transplanted spleens of mice treated with cyclosporine and BGO 136 are significantly smaller than those of the positive control mice (ANOVA<5).

As may be seen in this table, BGO 136 was more efficient than cyclosporine in preventing spleen growth following transplantation.

TABLE 2
INFLUENCE OF VARIOUS INHIBITORS ON SPLEEN WEIGH
AFTER TRANSPLANTATION

| | | splenomegaly | |
|---|---|---|---|
| Groups | Mice | mg/g of b. weight | suppression % |
| No treatment | 6 | 2.1 + 0.23 | 100 |
| | | 2.7 + 0.35 | |
| Positive | 8 | 7.1 + 1.11 | 0 |
| | | 7.7 + 1.65 | |
| BGO | 8 | 4.3 + 0.29 | 56 |
| AMP | 8 | 6.7 + 0.82 | 8 |
| Cyclosporine | 6 | 5.1 + 0.09 | 40 |

### CONCLUSION

The present invention showed a close correlation between inhibition of NTPDase activity and inhibition of immune responses of normal immune cells and neoplastic immune cells. All tested NTPDase inhibitors (i.e. BGO 136, erythrosin B, 8 Bus ATP, 8 Bus AMP and β-γ-Me ATP) decrease the NTPDase activity, and lymphocytic activity including proliferation and humoral response in normal T lymphocyte.

All the NTPDase inhibitors tested herein were shown to inhibit the growth of leukemic T lymphocyte cells (Jurkat cells) in culture.

As was seen in Table 1, the NTPDase activity in neoplastic lymphoid cells is 10 times higher than that in normal lymphocytes (exemplified herein by PBL). NTPDase is therefore inducible in tumoral lymphoid cells. It is reasonably predictable therefore that since NTPDase inhibitors were able to inhibit humoral response in PBL cells, it will a fortiari inhibit this response in tumoral lymphoid cells.

Mice were shown to be a reliable and predictive model for human in the use of immunosuppressive agents in graft transplant (i.e. cyclosporine). The graft transplant rejection mechanism therefore appears to be similar in mice and human. Furthermore, with regards particularly to NTPDase, as was shown in Benrezzak et al. 2000, human, mice (and pigs) have very similar biochemical profiles with regards to NTPDase activity and localization. Results presented herein obtained with mice may reasonably predict that similar results will be obtained in human for graft transplant and inhibition of immune cell activity involving NTPDase activity.

Although the present invention has been described hereinabove by way of preferred embodiments thereof, it can be modified, without departing from the spirit and nature of the subject invention as defined in the appended claims.

### REFERENCES

[1] Abbrachio, M. P. and Burnstock, G. (1998) Jpn. J. Pharmacol. 78, 113-145.
[2] Williams, M. and Jarvis, M. J. (2000) Biochem. Pharmacol. 59, 1173-1185.
[3] Drury, A. N. and Szent-Györgyi, A. (1929) J. Physiol. Lond. 68, 23-237.
[4] Bennet, D. W. and Drury, A. N. (1931) J. Physiol. Lond. 72,288-320.
[5] Holton, F. A. and Holton, P. (1954) J. Physiol. Lond. 126,124-140.
[6] Holton, P. (1959) J. Physiol. Lond. 145, 494-504.
[7] Burnstock, G. (1969) Pharmacol. Rev. 21, 247-324.
[8] Su, C.; Bevan, J. A. and Burnstock, G. (1971) Science 173, 336-338.
[9] Langer, S. Z. and Pinto, E. B. (1976) J. Pharmacol. Exp. Ther. 196, 697-713.
[10] Burnstock, G. (1976) J. Theor. Biol. 62, 491-503.
[11] Burnstock, G. (1971) Nature 229, 282-283.
[12] Burnstock, G. (1978) In: Cell membrane receptors for drugs and hormones: A multidisciplinary approach, (Straub, R. W. and Bolis, L.; Eds.), Raven Press, New York, pp. 108-118.
[13] Coade, S. B. and Pearson, J. D. (1989) Circ. Res. 65, 531-537.
[14] Beaudoin, a. r.; grondin, G.; enjyoji, K.; robson, S. C.; sévigny, J.; fischer, B. and gendron, F. P. (2000) in: Ecto-ATPases and related ectonucleotidases. (Vanduffel, L. and Lemmens, R.; Eds), Shaker Publishing B. V.; The Netherlands, pp. 125-135.
[15] Burnstock, G. (1972) Pharmacol. Rev. 24, 510-569.
[16] Lloyd, h. g. e. and stone, T. W. (1983) Proc. Physiol. Soc. 57p.
[17] White, T. D. (1988) Pharmacol. Ther. 38, 129-168.
[18] Williams, M. (1990) Ann. N.Y. Acad. Sci. 603, 93-107.
[19] Sneddon, P. and Burnstock, G. (1984) Eur. J. Pharmacol. 100, 85-90.
[20] Sneddon, P. and Westfall, D. P. (1984) J. Physiol. Lond. 347, 561-580.
[21] Burnstock, G. (1986) Prog. Brain Res. 68,193-203.
[22] Kirkpatrick, K. and burnstock, G. (1987) Eur. J. Pharmacol. 138, 207-214.
[23] Vizi, E. S. and Burnstock, G. (1988) Eur. J. Pharmacol. 158, 69-77.
[24] Westfall, D. P.; Sedaa, K. O.; Shinozuka, K.; Bjun, R. A. and Buxton, I. L. (1990) Ann. N.Y. Acad. Sci. 603, 300-310.
[25] Von Kugelgen, I. and Starke, K. (1994) Naunyn-Schmiedebergs Arch. Pharmacol. 350,123-129.
[26] Kennedy, C.; McLaren, G. J.; Westfall, T. D. and Sneddon, P. (1996) Ciba Foundation Symposium 198,223-235.
[27] Brock, J. A.; Bridgewater, M. and Cunnane, T. C. (1997) Br. J. Pharmacol. 120, 769-776.
[28] Richardson, P. J. and Brown, S. J. (1987) J. Neurochem. 48, 622-630.
[29] Ding, Y.; Cesare, P.; Drew, L.; Nikitaki, D. and Wood, J. N. (2000) J. Auton. Nerv. Syst. 81, 289-294.
[30] Hamilton, S. G. and McMahon, S. B. (2000) J. Auton. Nerv. Syst. 81, 187-194.
[31] Hamilton, S. G.; Wade, A. and McMahon, S. B. (1999) Br. J. Pharmacol. 126, 326-332.
[32] Unswoth, c. d. and johnson, R. G. (1990) Ann. N.Y. Acad. Sci. 603, 353-363.
[33] Uvnas, B. (1974) Life Sci. 14, 2355-2366.
[34] Roman, R. M.; wang, Y.; lidofsky, S. D.; feranchak, A. P.; Iomri, N.; scharschmidt, B. F. and fitz, J. G. (1997) J. Biol. Chem. 272, 21970-21976.
[35] Sprague, r. s.; ellsworth, M. L.; stephenson, A. H.; kleinhenz, M. E. and lonigro, A. J. (1998) Am. J. Physiol. 44, H1726-H1732.
[36] Sugita, m.; yue, Y. and foskett, J. F. (1998) EMBO J. 17, 898-908.
[37] Beaudoin, a. r.; grondin, G. and gendron, F. P. (1999) Prog. Brain Res. 120, 387-395.
[38] Taylor, a. l.; kudlow, B. A.; maus, K. L.; gruenert, D. C.; guggino, W. B. and schwiebert, E. M. (1998) Am. J. Pharmacol. 275, C1391-C1406.
[39] Wilson, p. d.; hovater, J. S.; casey, C. C.; fortenberry, J. A. and schwiebert, E. M. (1999) J. Am. Soc. Nephro. 10, 219-229.
[40] Schlosser, S. F.; Burgstahler, A. D. and Nathanson, M. H. (1996) Proc. Natl. Acad. Sci. USA 93, 9948-9953.
[41] Lidofsky, S. (1997) Hepatology 25, 778-779.
[42] Queiroz, g.; gebicke-haerter, P. J.; schobert, A.; starke, K. and Von kugelgen, L. (1997) Neurosci. 78, 1203-1208.
[43] Guthrie, p. b.; knappenberger, J.; segal, M.; bennett, M. V.; charles, A. C. and kater, S. B. (1999) J. Neurosci. 19, 520-528.
[44] Rojas, e.; pollard, H. B. and heldman, E. (1985) FEBS Letters 185, 323-327.
[45] Xu, y. p.; duarte, E. P. and forsberg, E. J. (1991) J. Neurochem. 56, 1889-1896.
[46] Weil-malherbe, h. and bone, A. D. (1958) Biochem. J. 70, 14-22.
[47] Marquardt, d. l.; gruber, H. E. and wasserman, S. I. (1994) Proc. Natl. Acad. Sci. USA 81, 6192-6196.
[48] Rapaport, e. and fontaine, J. (1989) Biochem. Pharmacol. 38, 4261-4266.
[49] Sperlagh, b.; hasko, G.; nemeth, Z. and vizi, E. S. (1998) Neurochem. Int. 33, 209-215.
[50] Fredholm, B. B.; Abbracchio, M. P.; G. Burnstock, G.; Daly, J. W.; Harden, T. K.; Jacobson, K. A.; Leff, P. and Williams, M. (1994) Pharmacol. Rev. 46,143-156.
[51] Tucker, A. L. and Linden, J. (1993) Cardiovasc. Res. 27, 62-67.
[52] Dubyak, G. R. and El-Moatassim, C. (1993) Am. J. Physiolgy 265, C577-C606.
[53] Stoop, R.; Thomas, S.; Rassedren, F.; Kawashima, E.; Buell, G.; Surprenant, A. and North, R. (1999) Mol. Pharmacol. 56, 973-981.
[54] Khakh, B. S.; Burnstock, G.; Kennedy, C.; King, B. F.; North, R. A.; Seguela, P.; Voigt, M. and Humphrey, P. P. A. (2001) Pharmacol. Rev. 53(1), 107-118.
[55] Weisman, G. A.; Turner, J. T.; Clarke, L. L.; Gonzalez, F. A, Otero, M.; Garrad, R. C. and Erb, L. (1997) in: Ecto-ATPases, (Plesner, L.; Kirley, T. L. and Knowles, A. F.; Eds.), Plenum Press. New York, USA, pp. 231-237.
[56] Evans, R. J.; Lewis, C.; Virgilio, C.; Lundstrom, K.; Buell, G.; Surprenant, A. and North, R. A. (1996) J. Physiol. Lond. 497, 413-422.
[57] Vulchanova, L.; arvidsson, U.; riedl, M.; wang, J.; buell, G.; surprenant, A.; north, R. A. and elde, R. (1996) Proc. Natl. Acad. Sci. USA 93, 8063-8067.
[58] Boarder, m. r. and hourani, s. m. o. (1998) Trends Pharmacol. Sci. 19, 99-107.
[59] Nicke, A.; Baumert, H. G.; Rettinger, J.; Eichele, A.; Lambrecht, G.; Mutschler, E. and Schmalzing, G. (1998) EMBO J. 17, 3016-3028.
[60] Fedan, J. S.; Dagirmanjian, J. P.; Attfield, M. A. and Chideskel, E. W. (1990) J. Pharmacol. Exp. Ther. 255, 46-51.
[61] Nakazawa, K.; Fujimori, K.; Takanakna, A. and Inoue, K. (1990) J. Physiol. Lond. 428, 257-272.
[62] Sela, D.; Ram, E. and Atlas, D. (1991) J. Biol. Chem. 266, 17990-17994.
[63] Berridge, M. J. and Irvine, R. F. (1989) Nature 341, 197-205.
[64] Gordon, J. L. (1986) Biochem. J. 233, 309-319.
[65] Surprenant, A.; Rassendren, F.; Kawashima, E.; North, R. A. and Buell, G. (1996) Science 272, 735-738.
[66] Ferrari, D.; Chiozzi, P.; Falzoni, S.; Dal Susino M.; Melchiorri, L.; Baricordi, O. R. and Di Virgilio, F. (1997) J. Immunol. 15, 1451-1458.
[67] Solle, M.; Labasi, J.; Perregaux, D. G.; Stam, E.; Petrushova, N.; Koller, B. H.; Griffiths, R. J. and Gabel, C. A. (2001) J. Biol. Chem. 276(1), 125-132.
[68] Sneddon, P.; Westfall, T. D.; Todorov, L. D.; Mihaylova-Todorova, S.; Westfall, D. P. and Kennedy, C. (1999) Prog. Brain Res. 120, 11-20.
[69] Kennedy, C. and Leff, p. (1995) Trends Pharmacol. Sci. 16, 168-174.
[70] North, R. A. (1996) Sem. Neurosci. 8,187-194.
[71] Torres, G.; Egan, T. and Voigt, M. (1999) J. Biol. Chem. 274, 6653-6659.
[72] Lewis, C.; Neidhart, S.; Holy, C.; North, R. A.; Buell, G. and Surprenant, A. (1995) Nature (Lond.) 377, 432-435.
[73] Cook, S. P.; Vulchanova, L.; Hargreaves, K. M.; Elde, R. and McCleskey, E. W. (1997) Nature (Lond.) 387, 505-508.
[74] Radford, K. M.; Virginio, C.; Surprenant, A.; North, R. A. and Kawashima, E. (1997) J. Neurosci. 17, 6529-6533.
[75] Vulchanova, L.; Riedi, M. S.; Shuster, S. J.; Buell, G.; Surprenant, A.; North, A. R. and Elde, R. (1997) Neuropharmacology 36, 1229-1242.
[76] Haines, W. R.; Torres, G. E.; Voigt M. M. and Egan, T. M. (1999) Mol. Pharmacol. 56, 720-727.
[77] Le, K. T.; Boue-Grabot, E.; Archambault V. and Séguéla, P. (1999) J. Biol. Chem. 274, 15415-15419.
[78] Torres, G. E.; Egan, T. M. and Voigt, M. M. (1998) Biochemistry 37, 4845-4851.
[79] Le, K. T.; Babinski, K. and Séguela, P. (1998) J. Neurosci. 18, 7152-7159.
[80] Hourani, S. M. O. and Hall, D. A. (1994) Trends Pharmacol. Sci. 15, 103-108.
[81] Communi, D.; Govaerts, C.; Parmentier, M. and Boeynaems, J. M. (1997) J. Biol. Chem. 272, 31969-31973.
[82] Leon, C.; Hechler, B.; Vial, C.; Leray, C.; Cazenave, J.-P. and Gachet, T. C. (1997) FEBS Lett. 403, 26-30.
[83] Nicholas, R. A.; Watt, W. C.; Lazarowski, E. R.; Li, Q. and Harden, T. K. (1996) Mol. Pharmacol. 50, 224-229.
[84] Boarder, M. R.; Weisman, G. A.; Turner, J. T. and Wilkinson, G. F. (1995) Trends Pharmacol. Sci. 16,133-138.
[85] Harden, T. K.; Boyer, J. L. and Nicholas, R. A. (1995) Ann. Rev. Pharmacol. Thoxicol. 35, 541-579.
[86] Yang, C. M.; Tsai, Y.-J.; Pan, S.-L.; Tsai, C.-T.; Wu, W.-b.; Chiu, C.-T.; Luo, S.-F. and Ou, J. T. (1997) Naunyn-Schmiedeberg's Arch. Pharmacol. 356, 1-7.
[87] Erb, L.; Lustig, K. D.; Sullivan, D. M.; Turner, J. T. and Weisman, G. A. (1994) Proc. Natl. Acad. Sci. USA 90, 10449-10453.
[88] Boyer, J. L.; Delaney, S. M.; Villanueva, D. and Harden, T. K. (2000). Mol. Pharmacol. 57, 805-810
[89] Hollopeter, G.; Jantzen, H. M.; Vincent, D.; Li, G.; England, L.; Ramakrishnan, V.; Yang, R. B.; Nurden, P.; Nurden, A.; Julius, D. and Conley, P. B. (2000) Nature 409, 202-207.
[90] Tu, M.-T.; Luo, S.-F.; Wang, C.-C.; Chien, C.-S.; Chiu, C.-T.; Lin, C.-C. and Yang, C. M. (2000) Br. J. Pharmacol. 129, 1481-1489.
[91] Erb, L.; Liu, J.; Ockerhausen, J.; Kong, Q.; Garrad, R. C.; Griffin, K.; Neal, C.; Krugh, B.; Santiago-Pérez, L. I.; Gonzalez, F. A.; Gresham, H. D.; Turner, J. T. and Weisman, G. A. (2001) J. Cell. Biol. 153, 491-501.
[92] Short, S. M.; Boyer, J. L. and Juliano, R. L. (2000) J. Biol. Chem. 275, 12970-12977.
[93] Zimmermann, H. (2000) Naunyn-Schmiedeberg's Arch. Pharmacol. 362, 299-309.
[94] Enjyoji, K.; Sévigny, J.; Lin, Y.; Fenette, P. S.; Christie, P. D.; Schulte am Esch II, J.; Imai, M.; Edelberg, J. M.; Rayburn, H.; Lech, M.; Beller, D. L.; Csizmadia, E.; Wagner, D. D.; Robson, S. C. and Rosenburg, R. D. (1999) Nature Med. 5,101-1017.
[95] LeBel, D.; Poirier, G. G.; Phaneuf, S.; St-Jean, P.; Laliberte, J.-F. and Beaudoin, A. R. (1980) J. Biol. Chem. 255, 1227-1233.
[96] Zimmermann, H. (1992) Biochem. J. 285, 345-365.
[97] Ziganshin, A. U.; Hoyle, C. H. V and Burnstock, G. (1994) Drug Dev. Res. 32,134-146.
[98] Plesner, L. (1995) Int. Rev. Cytol. 158,141-214.
[99] Beaudoin, A. R.; Sévigny, J. and Picher, M. (1996) In: Biomembranes Vol. 5. (Lee, A. G.; Ed.). JAI Press Inc. Greenwich, pp. 367-399.
[100] Vlajkovic, S. M.; Thome, P. R.; Hously, G. D.; Munoz, D. J. B. and Kendrick, I. S. (1998) Neuroreport 9, 1559-1565.
[101] Culic, O.; Sabolic, I. and Zanic-Grubisic, T. (1990) Biochim. Biophys. Acta 1030, 143-151.
[102] Meyerhof, 0. (1945) J. Biol. Chem. 157, 105-119.
[103] Kalckar, H. M. (1944). J. Biol. Chem. 153, 355-367.
[104] Ribeiro, J. M. C. (1987) Ann. Rev. Entomol. 32, 463-478.
[105] Ribeiro, J. M. C. (2000) Med. Vet. Entom. 14,142-148.
[106] Vasconcelos, E. G.; Nascimento, P. S.; Meirelles, M. N. L.; Verjovski-Almeida, S. and Ferreira, S. T. (1993) Mol. Biochem. Parasitol. 58, 205-214
[107] Anich, M.; Fanta, N.; Mancilla, M.; Kettlun, A, M, Velenzuela, M. A. and Traverso-Cori, A. (1990) Phytochemistry 29, 1411-1415.
[108] Thomas, C.; Sun, Y.; Naus, K.; Lloyd, A. and Roux, S. (1999) Plant Physiol. 119, 543-551.
[109] Bhargava, T.; Datta, S.; Ramachandran, V.; Ramakrishnan, R.; Roy, R. K.; Sankaran, K. and Subrahmanyam, Y. V. B. K. (1995) Curr. Sci. India 68, 293-300.
[110] Curdova, E.; Jechova, V. and Hostalek, Z. (1982) Folia Microbiol. 27, 159-166.
[111] Trilisenko, L. V.; Novotna, J.; Erban, V.; Behal, V. and Hostalek, Z. (1987) Folia Microbiol. 32, 402-410.
[112] Vasconcelos, E. G.; Ferraira, S. T.; De Carvalho, T. M. U.; De Souza, W.; Kettlun, A. M.; Mancilla, M.; Valenzuela, M. A. and Verjovski-Almeida, S. (1996) J. Biol. Chem. 271, 22139-22145.
[113] Berlutti, F.; Casalino, M.; Zagaglia, C.; Fradiana, P. A.; Visca, P. and Nicoletti, M. (1998) Inf. Immun. 66, 4957-4964.
[114] Yagi, K.; Arai, Y.; Kato, N.; Hirota, K. and Miura, Y. (1989) Eur. J. Biochem. 180, 509-513.
[115] Côté, Y.-P.; Picher, M.; St-Jean, P.; Béliveau, R.; Potier, M. and Beaudoin, A. R. 1991. Biochim. Biophys. Acta 1078, 187-191.
[116] Beaudoin, A. R.; Sévigny, J.; Grondin, G.; Daoud, S. and Levesque, F. P. (1997) Am. J. Physiol. 273: H673-H681.
[117] Sévigny, J.; Levesque, F. P.; Grondin, G. and Beaudoin, A. R. (1997). Biochim. Biophys. Acta 1334, 73-88
[118] Picher, M.; Côté, Y. P.; Béliveau, R.; Potier, M. and Beaudoin, A. R. (1993) J. Biol. Chem. 268, 4699-4703.
[119] Sévigny, J.; Picher, M.; grondin, G. and Beaudoin, A. R. (1997) Am. J. Physiol. 272, L939-L950.
[120] Sévigny, J.; grondin, G.; Gendron, F. P.; roy, J. and Beaudoin, A. R. (1998) Am. J. Physiol. 275, G473-G482.
[121] Leclerc, M.-C.; Grondin, G.; Gendron, F. P.; Sévigny, J. and Beaudoin, A. R. (2000) Arch. Biochem. Biophys. 377, 373-378.
[122] Sévigny, J.; Robson, S. C.; Waelkens, E.; Csizmadia, E.; Smith, R. N. and Lemmens, R. (2000) J. Biol. Chem. 275, 5640-5647.
[123] Zimmermann, H. and Braun, N. (1996) J. Auton. Pharmacol. 16, 397-400.
[124] Wang, T. F. and Guidotti, G. (1998) Brain Res. 790, 318-322.
[125] Maliszewski, C. R.; Delespesse, G. J. T.; Schoenborn, M. A.; Armitage, R. J.; Fanslow, W. C.; Nakajima, T.; Baker, E.; Sutherland, G. R.; Poindexter, K.; Birks, C.; Alpert, A.; Friend, D.; Gimpel, S. D. and Gayle III, R. B. (1994) J. Immunol. 153, 3574-3583.
[126] Benrezzak, O.; Grondin, G.; Sévigny, J.; Gendron, F. P.; Rousseau. E.; D'Orléans-Juste, P. and Beaudoin, A. R. (1999) Arch. Biochem. Biophys. 370, 314-322.
[127] Valenzuela, M. A.; Lopez, J.; Depix, M.; Mancilla, M.; Kettlun, A. M.; Catalan, L.; Chiong, M.; Garrido, J. and Traverso-Cori, A. (1989) Comp. Biochem. Physiol. 93B, 509-513.
[128] Papamarcaki, T. and Tsolas, O. (1990) Mol. Cell. Biochem. 97, 1-8.
[129] Magocsi, M. and Penniston, J. T. (1991) Biochim. Biophys. Acta 1070, 163-172.
[130] Pieber, M.; Valenzuela, M. A.; Kettlun, A. M.; Mancilla, M.; Aranda, E.; Collados, L. and Traverso-Cori, A. (1991) Comp. Biochem. Biophys. 100B, 281-285.
[131] Lemmens, R.; Kupers, L.; Sévigny, J.; Beaudoin, A. R.; Grondin, G.; Kittel, A.; Waelkens, E. and Vanduffel, L. (2000) Am. J. Physiol. 278, F978-F988.
[132] Handa, M. and Guidotti, G. (1996) Biochem. Biophys. Res. Comm. 218, 916-923.
[133] Schulte, J. A. E, Sévigny, J.; Kaczmarek, E.; Siegel, J. B.; Imai, M.; Koziak, K.; Beaudoin, A. R. and Robson, S. C. (1999) Biochemistry 38, 2248-2258.
[134] Smith, T. M.; Carl, S. A. L. and Kirley, T. L. (1999) Biochemistry 38, 5849-5857.
[135] Drosopoulos, J. H. F.; Broekman, M. J.; Islam, N.; Maliszewski, C. R.; Gayle, R. B. and Marcus, A. J. (2000) Biochemistry 39, 6936-6943.
[136] Grinthal, A. and Guidotti, G. (2000) Biochemistry 39, 9-16.
[137] Asai, T.; Miura, S.; Sibley, L. D.; Okabayashi, H. and Takeuchi, T. (1995) J. Biol. Chem. 270, 11391-11397.
[138] Kegel, B.; Braun, N.; Heine, P.; Maliszewski, C. R. and Zimmermann, H. (1997) Neuropharmacol. 36, 1189-1200.
[139] Stout, J. G. and Kirley, T. L. (1996) Biochemistry 35, 8289-8298.
[140] Wang, T. F.; Ou, Y. and Guidotti, G. (1998) J. Biol. Chem. 273, 24814-24821.
[141] Wang, T. F.; Rosenberg, P. A. and Guidotti, G. (1997) Brain Res. Mol. Brain Res. 47, 395-302.
[142] Sévigny, J.; Dumas, F. and Beaudoin, A. R. (1997) In: Ecto-ATPase. Recent progress on structure and function. (Plesner, L.; Kirley, T. L. and Knowles, A. F.; Eds.) Plenum Press. New York, 294 P.
[143] Chadwick, B. P. and Frischauf, A.-M. (1998) Genomics 50, 357-367.
[144] Schoenborn, M. A.; Jenkins, N. A.; Copeland, N. G.; Gilbert, P. J.; Gayle III, R. B. and Maliszewski C. R. (1998) Cytogenet. Cell Genet. 81, 287-289.
[145] Zimmermann, H.; Beaudoin, A. R.; Bollen, M.; Goding, J. W.; Guidotti, G.; Kirley, T. L.; Robson, S. C. and Sano, K. (2000). In: Ecto-ATPases and related ectonucleotidases. (Vanduffel, L. and Lemmens, R.; Eds) Shaker Publishing B. V.; The Netherlands. Pp 1-8.
[146] Côté, Y. P.; Filep, J. G.; Battistini, B.; Gauvreau, J.; Sirois, P. and Beaudoin, A. R. (1992) Biochim. Biophys. Acta 1139,133-42.
[147] Kirley, T. L. (1997) J. Biol. Chem. 272, 1076-1081.
[148] Marcus, A. J.; Broekman, M. J.; Drosopoulos, J. H. F.; Islam, N.; Alyonycheva, T. N.; Safier, J. B.; Hajjar, K. A.; Posnett, D. N.; Schoenborn, M. A.; Schooley, K. A.; Gayle III, R. B. and Maliszewski, C. R. (1997) J. Clin. Invest. 99, 1351-1360.
[149] Koziak, K.; Sévigny, J.; Robson, S. C.; Siegel, J. B. and Kaczmarek, E. (1999) Thromb. Haemo. 82, 1538-1544.
[150] Kansas, G. S.; Wood, G. S. and Tedder, T. F. (1991) J. Immunol. 146, 2235-2244.
[151] Frassetto, S. S.; Dias, R. D. and Sarkis, J. J. F. (1993) Mol. Cell. Biochem. 129, 47-55.
[152] Marcus, A. J. (1996) In: Disorders of Hemostasis, chap. 4 (O. D. Ratnoff and C. D. Forbes, ed.), pp. 79-137, Philadelphia, U.S.A.
[153] Hirota, K.; Saski, N.; Yagi, K. and Miura, Y. (1987) Thromb. Res. 45, 201-209.
[154] Kaczmarek, E.; Koziak, K.; Sévigny, J.; Siegel, J. B.; Anrather, J.; Beaudoin, A. R.; Bach, F. H. and Robson, S. C. (1996) J. Biol. Chem. 271, 33116-33122.
[155] Koyamada, N.; Miyatake, T.; Candinas, D.; Hechenleither, P.; Siegel, J.; Hancock, W. W.; Bach, F. H. and Robson, S. C. (1996) Transplantation 62, 1739-1743.
[156] Imai, M.; Takigami, K.; Guckelberger, O.; Enjyoji, K.; Neal Smith, R.; Lin, Y.; Csizmadia, E.; Sévigny, J.; Rosenberg, R. D.; Bach, F. H. and Robson, S. C. (1999) Mol. Med. 5, 743-752.
[157] Braun, N.; Zhu, Y, Krieglstein, J.; Culmsee, C. and Zimmermann, H. 1998. J. Neurosci. 18, 4891-4900.
[158] Rongen, G. A.; Floras, J. S.; Lender, J. W.; Thien, T.; Smits, P. (1997) Clin. Sci. 92, 13-24.
[159] Di Virgilio, F.; Bronte, V.; Collavo, D. and Zanovello, P. (1989) J. Immunol. 143, 1955-1960.
[160] Filippini, A.; Taffs, R. E.; Agui, T. and Sitkovsky, M. V. (1990) J. Biol. Chem. 265, 334-340.
[161] Antonysamy, M. A.; Moticka, E. J. and Ramkumar, V. (1995) J. Immunol. 155, 2813-2821.
[162] Krishnaraj, R. (1992) Cell Immunol. 141, 306-322.
[163] Krishnaraj, R. (1992b) Cell Immunol. 144, 11-21.
[164] Bajpai, A. and Brahmi, Z. (1993) Cell. Immunol. 148,130-143.
[165] Dombrowski, K. E.; Cone, J. C.; Bjorndahl, J. M. and Phillips, C. A. (1995) Cell. Immunol. 160, 199-204.
[166] Correale, P.; Giuliano, M.; Tagliaferri, P.; Guarrusi, R.; Caraglia, M.; Marinetti, M. R.; Iezzi, T.; Bianco, A. R. and Procoplo, A. (1995) Res. Comm. Mol. Pathol. Pharmacol. 87, 67-69.
[167] Imai, M.; Goepfert, C.; Kaczmareck E. and Robson, S. C. (2000) Biochem. Biophys. Res. Comm. 270, 272-278.
[168] Clifford, E. E.;. Martin, K. A.; Dalal, P.; Thomas, R. and Dubyak, G. R. (1997) Am. J. Physiol. 42, C973-C987.
[169] Dzhandzhugazyan, K. N.; Kirkin, A. F.; Straten, P. T. and Zeuthen, J. (1998) FEBS Left. 430, 227-230.
[170] Gendron, F. P.; Halbfinger, E.; Fischer, B.; Duval, M.; D'Orléans-Juste, P. and Beaudoin, A. R. (2000a) J. Med. Chem. 43, 2239-2247.
[171] Gendron, F. P.; Halbfinger, E.; Fischer, B.; and Beaudoin A. R. (2000) In: Purine and pyrimidine metabolism in man X (Eds. E. Zoref-Shani and 0. Sperling). Adv. Exp. Med. Biol. 486,119-123.
[172] Picher, M.; Sévigny, J.; D'Orléans-Juste, P. and Beaudoin, A. R. (1996) Biochem. Pharmacol. 51, 1453-1460.
[173] Fischer, B.; Chulkin, A.; Boyer, J. L.; Harden, K. T.; Gendron, F. P.; Beaudoin, A. R.; Chapal, J.; Hillaire-Buys, D. and Petit, P. (1999) J. Med. Chem. 42, 3636-3646.
[174] Halbfinger, E.; Major, D. T.; Ritzman, M.; Ubl, J. J.; Reiser, G.; Boyer, J. L.; Harden, K. T. and Fischer, B. (1999) J. Med. Chem. 42, 5325-5337.
[175] Fischer, B.; Kabha, E.; Gendron, F. P. and Beaudoin, A. R. (2000) Nucleosides, Nucleotides & Nucleic Acid 19, 1033-1054.
[176] Bültmann, R.; Wittenburg, H.; Pause, B.; Kurz, G.; Nickel, P. and Starke, K. (1996) Naunyn-Schmiedeberg's Arch. Pharmacol. 354, 498-504.
[177] Tuluc, F.; Bültmann, R.; Glanzel, M.; Wilhelm Frahm, A. and Starke, K. (1998) Naunyn-Schmiedeberg's Arch. Pharmacol. 357, 111-120.
[178] Wittenburg, H.; Bültmann, R.; Pause, B.; Ganter, C.; Kurz, G. and Starke. K. (1996) Naunyn-Schmiedeberg's Arch. Pharmacol. 354, 491-497.
[179] Bültmann, R.; Pause, B.; Wittenburg, H.; Kurz, G. and Starke, K. (1996) Naunyn-Schmiedeberg's Arch. Pharmacol. 354, 481-490.
[180] Parr, C. E.; Sullivan, D. M.; Paradiso, A. M.; Larowski, E. R.; Burch, L. H.; Olsen, J. C.; Erb, L.; Weisman, G. A.; Boucher, R. C. and Turner, J. T. (1994) Proc. Nat. Acad. Sci. USA 91, 3275-3279.
[181] Vlajkovic,. S. M.; Hously, G. D.; Greenwood, D. and Thorne PR. (1999) Mol. Brain Res. 73, 85-92.
[182] Zimmermann, H. (1999) Nat. Med. 5, 987-988.
[183] Bergfeld, g. r. and forrester, T. (1992) Cardiovas. Res. 26, 40-47.
[184] Knofler, r.; Weissbach, G. and Kuhlisch, E. (1997) Am. J. Hematol. 56, 259-265.
[185] Detwiler, t. c. and feinman, R. D. (1973) Biochemistry 12, 2462-2468.
[186] Li, b. y. and li, W. H. (1998) Acta Pharmacol. Sinica 19, 383-386.
[187] Bodin, p. and burnstock, G. (1995) Experientia 51, 256-259.
[188] Bodin, p.; milner, P.; winter, R. and burnstock, G. (1992) Proc. R. Soc. Lond. 247,131-135.
[189] Schini, v. b.; hendrickson, H.; heublein, D. M.; burnett, J. C. and vanhoutte, P. M. (1989) Eur. J. Pharmacol. 165, 333-334.
[190] Bodin, p. and burnstock, G. (1996) J. Cardiovasc. Pharmacol. 27, 872-875.
[191] Sedaa, k. o.; bjur, R. A.; schinozuka, K. and westfall, D. P. (1990) J. Pharmacol. Exp. Ther. 252, 1060-1067.
[192] Yang, s.; cheek, D. J.; westfall, D. P. and buxton, I. L. (1994) Circ. Res. 74, 401-407.
[193] Vizi, e. s.; sperlagh, B. and burnstock, G. (1992) Neurosci. 50, 455-465.
[194] Katsuragi, t.; tokunaga, T.; ogawa, S.; soejima, O.; sato, C. and furukawa, T. (1991) J. Pharmacol. Exp. Ther. 259, 513-518.
[195] Katsuragi, t.; tamesue, S.; sato, C.; sato, Y. and furukawa, T. (1996) Naunyn-Schmiedebergs Arch. Pharmacol. 354, 796-799.
[196] Tamesue, s.; sato, C. and katsuragi, T. (1998) Naunyn-Schmiedebergs Arc. Pharmacol. 357, 240-244.
[197] Gendron F. P., Benrezzak, O., Krugh, B. W., Kong, Q., Weisman, G. A., and Beaudoin A. R. 2002. Purine signalling and potential new therapeutic approach: possible outcome of NTPDase inhibition. Current Drug Targets 3(3): 229-245.
[198] Nahum, V., Zundorf, G., Reiser, G., Levesque, S. A., Beeaudoin, A. R., Fischer, B. 2002. 5′-O-(1-Boranotriphosphate)-Adenosine Derivatives as Novel P2Y₁-Receptor Agonists. (Accepted J. Med. Chem.)

1. A method of screening for a compound useful in the treatment of a disease or condition characterized by an immune cells disorder, wherein said cell expresses NTPDases, said method comprising the steps of
contacting a candidate compound with ecto-nucleoside triphosphate diphosphohydrolase (NTPDase),
wherein the candidate compound is selected if the activity of said NTPDase is reduced in the presence of the candidate compound as compared to that in the absence thereof.

2. A method as defined in claim 1, wherein said contacting of said candidate compound with said NTPDase is performed in an immune cell selected from the group consisting in normal T lymphocyte, normal B lymphocyte, normal NK cell, normal macrophage, normal monocyte, Jurkat cell, Raji cell, Ramos cell, MonoMac™ cell, K562 cell and U937 cell.

3. A method as defined in claim 2, wherein said immune cell is a T lymphocyte.

4. A method as defined in claim 2, wherein said immune cell is a B lymphocyte.

5. A method as defined in claim 2, wherein said immune cell is a Jurkat cell.

6. A method as defined in claim 2, wherein said immune cell is a Raji Cell.

7. A method as defined in claim 2, wherein said immune cell is a Ramos cell.

8. A method as defined in claim 2, wherein said immune cell is a MonoMac™ cell.

9. A method as defined in claim 2, wherein said immune cell is a K562 cell.

10. A method as defined in claim 2, wherein said immune cell is a U937 cell.

11. A method as defined in claim 2, wherein said immune cell is a NK cell.

12. A method for inhibiting an immune cell activity in a mammal, comprising targeting immune cells with an effective amount of a NTPDase inhibitor.

13. A method as recited in claim 12, wherein said cells are normal lymphocytes.

14. The method as recited in claim 13, wherein said normal cells are normal T lymphocytes.

15. The method as recited in claim 14, wherein said activity is the T lymphocyte proliferation.

16. The method as recited in claim 13, wherein said normal lymphocytes are normal B lymphocyte.

17. The method as recited in claim 12, wherein said activity is the production of antibodies.

18. A method as recited in claim 12, wherein said cells are neoplastic lymphocytes.

19. The method as recited in claim 18, wherein said neoplastic lymphocytes are neoplastic T lymphocytes.

20. The method as recited in claim 19, wherein said neoplastic T lymphocytes are Jurkat cells.

21. The method as recited in claim 12, wherein said activity is induced by organ or tissue transplant.

22. The method as recited in claim 12, wherein said activity is induced by an allergen.

23. The method as recited in claim 12, wherein said activity is induced in autoimmune diseases.

24. A method as recited in claim 12, wherein said NTPDase inhibitor is selected from the group consisting of BGO 136, erythrosin B, nucleotide or nucleotide derivative including AMP, 8 Bus-AMP and 8 Bus-ATP, and analogues thereof.

25. The method as recited in claim 12, wherein said inhibitor is BGO136 or a BGO136 analogue.

26. The method as recited in claim 12, wherein said inhibitor is AMP or an AMP analogue.

27. The method as recited in claim 12, wherein said inhibitor is 8 Bus-AMP.

28. The method as recited in claim 12, wherein said inhibitor is ATP or an ATP analogue.

29. The method as recited in 12, wherein said inhibitor is 8 Bus-ATP.

30. The method as recited in claim 12, wherein said inhibitor is erythrosine B or an erytbrosin B analogue.

31. A method to prevent or reduce the risk of rejection of transplanted tissue or organ, comprising administering to said animal an effective amount of NTPDase inhibitor.

32. A method as recited in claim 31, wherein said NTPDase inhibitor is selected from the group consisting of BGO 136, erythrosin B, nucleotide or nucleotide derivative including AMP, 8 Bus-AMP and 8 Bus-ATP, and analogues thereof.

33. The method as recited in claim 32, wherein said inhibitor is BGO136 or a BGO136 analogue.

34. The method as recited in claim 32, wherein said inhibitor is AMP or an AMP analogue.

35. The method as recited in claim 32, wherein said inhibitor is 8 Bus-AMP.

36. The method as recited in claim 32, wherein said inhibitor is ATP or an ATP analogue.

37. The method as recited in claim 32, wherein said inhibitor is 8 Bus-ATP.

38. The method as recited in claim 32, wherein said inhibitor is erythrosine B or an erythrosin B analogue.

39. The method as recited in claim 32, wherein said inhibitor is BGO 136.

40. Composition for use as an immunosuppressive agent in graft transplant comprising an effective amount of BGO 136 or BGO 136 analogue in a pharmaceutically acceptable carrier.

41. Use of a BGO 136 or BGO 136 analogue in the making of a medicament for use as an immunosuppressive agent in graft transplant.
